(19) United States
(12) Patent Application Publication
(10) Pub. No.: US 2026/0098856 A1
(43) Pub. Date: Apr. 9, 2026

(54) MOTION BLUR MICROSCOPY

(71) Applicant: CASE WESTERN RESERVE UNIVERSITY, Cleveland, OH (US)

(72) Inventors: Utku Goreke, Shaker Heights, OH (US); Brandon Shipley, Cleveland, OH (US); Ayesha Gonzales, Cleveland, OH (US); Michael Hinczewski, Cleveland, OH (US); Umut A. Gurkan, Shaker Heights, OH (US)

(21) Appl. No.: 19/352,707

(22) Filed: Oct. 8, 2025

Related U.S. Application Data
(60) Provisional application No. 63/704,794, filed on Oct. 8, 2024.

(51) Int. Cl. G01N 33/49; G01N 15/14; G01N 15/1433

(52) U.S. Cl. CPC G01N 33/4915; G01N 15/1433; G01N 15/1459; G01N 15/1484; G01N 2015/1402; G01N 2015/1493

(57) ABSTRACT

A method of determining one or more hemodynamic properties or cell type of a blood sample includes obtaining a set of one or more motion blur microscopy (MBM) images of the blood sample flowing through a microchannel of a microfluidic device and inputting the set of one or more MBM images into one or more machine learning models trained to: generate a segmentation map based on based on the set of one or more MBM images, the segmentation map including a adhered pixels or background pixels; the adhered pixels corresponding to a pixel of the MBM images belonging to an adhered object in the microchannel and with remaining pixels being background pixels; generate a cell classification of the adhered pixels based on a physical property of the cells; and detect type or hemodynamic properties of the cells based on the cell classification.

### RELATED APPLICATION

This application claims priority from U.S. Provisional Application No. 63/704,794, filed Oct. 8, 2024, the subject matter of which is incorporated herein by reference in its entirety.

### GOVERNMENT FUNDING

This invention was made with government support under 1552782 and 1651560 awarded by the National Science Foundation, and HL133574 and HL152643 awarded by the National Institutes of Health. The government has certain rights in the invention.

### BACKGROUND

Cellular interactions, including cell adhesion, migration, and chemo-taxis, are important in investigating the mechanisms of diseases including cancer, thrombosis, inflammatory diseases, anemia, and vasculopathy. In vitro cellular imaging techniques for hematology generally require the use of an aqueous buffer, which dilutes the sample and allows transmission of light for imaging. However, buffer solutions replace the original whole blood medium, potentially affecting the biological mechanisms under investigation. For instance, plasma proteins facilitate the interaction of red blood cells with endothelial cells, and red blood cells induce margination of leukocytes and platelets to the vascular wall. Comprehensively understanding these phenomena requires a physiologically realistic approach that includes the presence of whole blood. Therefore, intravital methods remain the gold standard for studies of important dynamic processes associated with cellular interactions. Intravital methods include multi- and single-photon microscopy, confocal microscopy, Brillouin spectroscopy combined with light microscopy, lightsheet microscopy, and endomicroscopy. However, intravital microscopy methods are highly costly, and require intensive effort in both setup and analysis, and therefore have limited applicability for the broader research community. Total internal reflection fluorescence microscopy is a potential in vitro alternative for visualizing cellular interactions under whole blood flow (and examples with buffer flow already exist). But fluorophore labeling in whole blood can be challenging. Finally, laser optical imaging has been used for obtaining the number of platelet interactions that occur with a protein substrate in a microfluidic channel, but this method is limited to a single numeric output (intensity of light scattered over time) and has additional experimental setup complexity.

### SUMMARY

This disclosure provides systems and methods that employ motion blur microscopy (MBM) and machine learning to classify blood cells and hemodynamic properties of blood cells. Imaging and characterizing the dynamics of cellular adhesion in blood samples is of fundamental importance in understanding biological function. In vitro microscopy methods are widely used for this task but typically require diluting the blood with a buffer to allow for transmission of light. However, whole blood provides crucial signaling cues that influence adhesion dynamics, which means that conventional approaches lack the full physiological complexity of living microvasculature.

We found that we can reliably image cell interactions in microfluidic channels during whole blood flow by motion blur microscopy (MBM) in vitro and automate image analysis using machine learning. With our automated analysis, we can process MBM images in an accurate and high throughput manner. Adhered cells can be identified, and morphological features, such as the size and eccentricity of each individual cell can be extracted. MBM videos enable studying the dynamics of cells on the individual level and allow us to determine kinetic properties like adhesion durations and average velocities (for the case of cells that roll or migrate while on the surface). The individual cell data can be aggregated to produce group statistics, such as distributions of morphological features and dynamic quantities, or mean squared displacements. Importantly, the system and method described herein allows us to identify and analyze the properties of hundreds of thousands of cells. MBM can be generalizable to studies of various diseases, including cancer, blood disorders, thrombosis, inflammatory and autoimmune diseases, as well as provide rich datasets for theoretical modeling of adhesion dynamics.

In some embodiments, the system includes a microfluidic device having at least one microchannel through which a fluid sample including cells, such as an undiluted whole blood sample, flows, an imaging system configured for generating one or more sets of motion blur microscopy (MBM) images of cells of interest in the microchannel when the fluid sample containing cells is passed therethrough, one or more non-transitory computer-readable storage media including instructions, and one or more processors coupled to the one or more non-transitory computer-readable storage media. The one or more processors are configured to execute the instructions to access the set of one or more MBM images generated by the imaging system, input the set of the one or more MBM images into one or more machine learning models trained to generate a segmentation mask based on the set of one or more MBM images, the segmentation mask including adhered pixels or background pixels, the adhered pixels corresponding to a pixel of the MBM images belonging to an adhered object in the microchannel and with remaining pixels being background pixels, generate a cell classification of the adhered pixels based on a physical property of the cells, and detect a cell type or hemodynamic property of the cells based on the cell classification.

In some embodiments, the microfluidic device includes a housing with at least one microchannel defining at least one cell adhesion region. The at least one cell adhesion region can be provided with at least one capturing agent that adheres a cell of interest to a surface of the at least one microchannel when a fluid sample containing cells is passed through the at least one microchannel.

In some embodiments, the imaging system includes a camera configured to obtain a plurality of MBM images of the fluid sample flowing through the cell adhesion region.

In some embodiments, the processor is configured to identify groups of pixels in the MBM image corresponding to adhered cells and generate of the cell classification based on cell size.

In some embodiments, the cell classification determines the type of cell.

In some embodiments, the one or more machine-learning models include a segmentation model and a classification model.

In some embodiments, the segmentation model is trained by selecting one or more MBM images as training/validation images, creating a labeled training/validation mask of the training/validation images by labeling each pixel as adhered or background, splitting each training/validation mask and training/validation images into tiles of pixels, and applying data augmentation to produce unique orientations of the tiles.

In some embodiments, the segmentation model labels pixels from the MBM images corresponding to adhered objects and groups together neighboring labeled pixels.

In some embodiments, processor uses groups of labeled pixels to classify the cell type using a size threshold or a specifically trained classification neural network.

In some embodiments, the processor further classifies cell type by using cell morphological properties, such as cell size and eccentricity, as well as cell dynamic properties, such as cell adhesion duration or mean velocity.

Other embodiments described herein relate to a method of determining one or more hemodynamic properties or cell type of a fluid sample including cells, such as a blood sample, and particularly an undiluted whole blood sample. The method includes obtaining a set of one or more motion blur microscopy (MBM) images of the fluid sample flowing through a microchannel of a microfluidic device. The set of one or more MBM images is inputted into one or more machine learning models trained to: generate a segmentation mask based on based on the set of one or more MBM images the segmentation mask including adhered pixels and background pixels, the adhered pixels corresponding to a pixel of the MBM images belonging to an adhered object in the microchannel and the remaining pixels being background pixels; generate a cell classification of the adhered pixels based on a physical property of the cells; and detect a cell type or hemodynamic properties of the cells based on the cell classification.

In some embodiments, the microfluidic device includes a housing with at least one microchannel defining at least one cell adhesion region. The at least one cell adhesion region can be provided with at least one capturing agent that adheres a cell of interest to a surface of the at least one microchannel when a fluid sample containing cells is passed through the at least one microchannel.

In some embodiments, the machine learning model is configured to identify groups of pixels in the MBM image corresponding to adhered cells and generate a cell classification based cell size.

In some embodiments, the cell classification determines the cell type.

In some embodiments, the one or more machine-learning models include a segmentation model and a classification model.

In some embodiments, the segmentation model is trained by selecting one or more MBM images as training/validation images, creating a labeled training/validation mask of the training/validation images by labeling each pixel as adhered or background, splitting each training/validation mask and training/validation images into tiles of pixels, and applying data augmentation to produce unique orientations of the tiles.

In some embodiments, the segmentation model labels pixels from the MBM images corresponding to adhered objects and groups together neighboring labeled pixels.

In some embodiments, the machine learning model uses groups of labeled pixels to classify the cell type using a size threshold or a specifically trained classification neural network.

In some embodiments, the classification model further classifies cell type by using cell morphological properties as well as cell dynamic properties.

### BRIEF DESCRIPTION OF THE DRAWINGS

FIG. 1 is a flow diagram depicting an example of a method described herein.

FIGS. 2(A-D) illustrate motion blur microscopy (MBM). A Moving objects in the foreground obstruct the stationary objects in the background. B By adjusting the exposure, moving objects are blurred to obtain a clear view of the background. C The same principle is applied to microscale whole blood flow. Shown are three views of whole blood flow in the same microscopic field with different microscope settings. Increasing the camera exposure (integration time) helps blur the foreground, which consists of non-adhered (flowing) cells. Long exposure results in excess brightness which is then compensated by a reduction of the light source voltage to obtain a clear view of the adhered cells in the background. Yellow arrows show the flow direction. The shear rate of the flow is 50 s⁻¹ which is enough to induce the blur at 1200 ms integration time. D Schematic illustration of the microfluidic channel, with clearly visible adhered cells and blurry flowing cells.

FIGS. 3(A-E) illustrate MBM allows capturing cellular interactions on endothelial layers and PBS dilution diminishes the sickle RBC adhesion events. A Human umbilical vein endothelial cell (HUVEC) layer on the microfluidic device surface without blood flow. B Original MBM images of sickle red blood cells (RBCs) on the endothelial surface, shown without gray histogram adjustment. C MBM image shown in inset (b) after stretching the histogram to its limits. D Aberrant cellular aggregations in diluted flow. E Under whole blood flow the number of adhered HbSS RBCs was significantly greater than HbAA RBCs. The number of adhered RBCs did not increase after extended flow duration. On the other hand, the dilution of the HbSS whole blood resulted in similar adhesion in HbAA and HbSS samples. Moreover, extended flow duration significantly increased the RBC adhesion through aberrant cellular interactions such as clumping. Red circles denote locations of adhered RBCs. The scale bar applies to all images. n=3, biological replicates, p-values are calculated with one-tailed Mann-Whitney with ties and continuity correction.

FIGS. 4(A-E) illustrate cartoon diagram of automated analysis pipeline and example objects. A The pipeline is conducted in two distinct phases. In the first phase (segmentation network), groups of adhered pixels that may correspond to an adhered cell are identified. In the second phase (size thresholding), these groups are classified by cell type. B-E Four examples of adhered objects that might appear in MBM images. (B, C) show cells of interest, while (D, E) show non-relevant adhered regions.

FIGS. 5(A-B) illustrate size and eccentricity distributions under whole blood flow. Joint probability distributions for the size and eccentricity of (A) objects adhered to laminin functionalized channels from 174 images (N=162,207) and (B) objects adhered to E-selectin functionalized channels from 2 videos (N=5919). Marginal distributions of size and eccentricity are shown on the top and right axes respectively. Dotted vertical lines indicate the size threshold used for classification, with objects above the threshold corresponding to sRBCs in panel (A) or CAR-T cells in panel (B).

FIGS. 6(A-D) illustrate validation and benchmarking of MBM. A We establish inter-experimenter reproducibility for adhered sickle red blood cell (sRBC) counts for different experimental durations, carried out by two different researchers. Shaded regions around each line are one standard deviation, dots represent the average count for each researcher. The cell adhesion results show no significant difference when two different experimenters perform MBM using the aliquots of the same patient sample. B We establish the sensitivity of MBM for adhered sRBC counts. For all but one data point, the coefficient of variation is <25%, an important benchmark for the precision of bioanalytical methods. Finally, we establish the accuracy of MBM in counting (C) sRBCs adhered to laminin and (D) CAR-T cells adhered to E-selectin, respectively. There is a strong agreement between automated and human counts in both cases, as indicated by the R² value being close to one.

FIGS. 7(A-B) illustrate example adhered cell trajectories. Shown are (A) sickle red blood cell (sRBC) motion on laminin and (B) CAR-T cell motion on E-selectin. Generally speaking, the longer a cell is adhered, the smaller the displacement of the cell in the direction of the flow. CAR-T cells tend to have higher motility relative to sRBCs.

FIGS. 8(A-B) illustrate probability distributions of adhesion durations. Shown are (A) sickle red blood cell (sRBC) adhesion to laminin (N=14,229) and (B) CAR-T cell adhesion to E-selectin (N=7671).

FIGS. 9(A-B) illustrate relationships of adhesion duration and morphological features of adhered cells under whole blood flow. Average eccentricity vs. adhesion duration for (A) sickle red blood cell (sRBC) adhesion to laminin under normoxic conditions (N=14,229), and (B) CAR-T cell adhesion to E-Selectin (N=5919). Dotted lines show 95% confidence intervals for the line of best fit. The blue histograms show the distribution of average eccentricities. There is a significant negative relationship between average eccentricity and adhesion duration for sickle RBCs, but not for CAR-T cells (p=0.03, p=0.07, each panel respectively, linear regression). Blue dots represent means, and whiskers for each scatter point are one standard deviation.

FIGS. 10(A-C) illustrate velocities of cells under whole blood flow. Each row shows a velocity probability distribution (left) and a corresponding average velocity versus adhesion duration plot (right). Three cases are depicted: a Parallel to the flow direction for sickle red blood cell adhesion (sRBC) to laminin (N=14,229). The probability distribution shows that a large majority of adhesion events have near-zero velocities. B Perpendicular to the flow direction for sickle red blood cell (sRBC) adhesion to laminin. Because this is the perpendicular to flow direction, we expect the distribution to approach that of a random walk, and the average velocities to be near zero. C Parallel to flow for CAR-T cell adhesion to E-selectin (N=7671). When comparing this row to (A), we see that CAR-T cells adhering to E-selectin tend to have larger parallel to flow velocities. Perpendicular to flow analysis of CAR-T cells is not shown, but the results are similar to those of (B). Error bars denote one standard deviation.

FIGS. 11(A-E) illustrate the effect of CD19 activation on the motility of CAR-T cells adhered to E-selectin. Velocity distribution of CAR-T cells for control (A) and CD19 activated (B) systems, with color coding highlighting different velocity ranges. C Activation leads to significantly increased mean rolling velocity among the rolling cells (1.85±0.06 μm/s, n=1247 vs. 2.77±0.06 μm/s, n=2408, data is mean±95% CI, p=6×10⁻⁸⁵, black bars denote the means, t-test with two-tailed distribution without adjustments). Trajectories of adhered cells for the control (D) and activated (E) cases, labeled by their respective velocity ranges according to the same color scheme as in the distribution panels.

FIG. 12 illustrates the architecture of the phase 1 segmentation network. We show an example of input and output for sickle red blood cell adhesion to laminin, as well as manual classification for comparison. The groups of adhered pixels found by the segmentation network appear to match manual classification well. Conv=2D Convolutional. BN=Batch Normalization. MP=Max Pooling. ConvT=2D Convolutional Transpose. Concat=Concatenation.

FIGS. 13(A-B) illustrate phase 2 classification network architecture A) Schematic of the architecture for the phase 2 classification network. Only the final four layers are trainable. Conv=2D Convolutional. MP=Max Pooling. D=Dense. B) Cartoon representation of MBM with automated analysis for analysis of multiple cell types. Classification now requires a neural network.

FIG. 14 illustrates CAR-T cell/sickle red blood cell adhesion to P-selectin size vs. eccentricity distribution. Hexplot showing the joint distribution of size and eccentricity for CAR-T/red blood cell adhesion to P-selectin for 947 consecutive video frames (N=82572). Projections on each axis show the marginal distributions of size and eccentricity alone. Note that the distribution peak at large areas corresponds to both CAR-T and red blood cells, and so the two types cannot be distinguished via size/eccentricity characteristics alone.

FIGS. 15(A-C) illustrates validation of MBM with automated analysis with more than one cell type a) F₁ score vs. confidence threshold plot. There is a range of confidence thresholds that produce acceptable F₁ scores. B) Confusion matrix for the classification network on a subset of groups of adhered pixels (N=885), for a confidence threshold of 0.67. The true positive rate is large for quantified cells. C) Comparison between automated and human counts of CAR-T cell adhesion to P-selectin. The R² value indicates good agreement between the two counts.

FIG. 16 illustrates adhesion duration profiles for red blood cells (RBCs) with HbAA and HbSS genotype, and under normoxic and hypoxic conditions during whole blood flow. The adhesion duration of RBCs shows a power law distribution at short times (red lines indicate estimated scaling exponents). HbSS-containing sickle RBCs establish longer-lasting bonds more than HbAA-containing RBCs, but short-duration adhesion events show a resemblance between genotypes. Hypoxia increases the long-duration adhesion events in only sickle whole blood samples.

FIG. 17 illustrates paths of two crawling leukocytes on an E-selectin surface under whole blood flow. We used whole blood from a healthy subject collected in a heparin tube and we did not perform any preprocessing. Path color denotes how much time is spent during crawling. The initial locations shown are random.

FIG. 18 illustrates replenishing Ca++ increases the number of Jurkat cells adhered to E-selectin significantly. Blood sample collection with EDTA tubes prevents coagulation by calcium chelation, but calcium is crucial for leukocyte activity. Ca++ is replenished by resuspending the mixture of HbAA RBCs and Jurkats at 40% hematocrit in Hank's buffer containing calcium. Control is resuspension in PBS without calcium. n=3. p-value is calculated with, SVTLBM-8BMMJT.

### DETAILED DESCRIPTION

This disclosure provides systems and methods that employ motion blur microscopy (MBM) and machine learning to classify blood cells and hemodynamic properties of blood cells. Imaging and characterizing the dynamics of cellular adhesion in blood samples is of fundamental importance in understanding biological function. In vitro microscopy methods are widely used for this task but typically require diluting the blood with a buffer to allow for transmission of light. However, whole blood provides crucial signaling cues that influence adhesion dynamics, which means that conventional approaches lack the full physiological complexity of living microvasculature.

We found that we can reliably image cell interactions in microfluidic channels during whole blood flow by motion blur microscopy (MBM) in vitro and automate image analysis using machine learning. With our automated analysis, we can process MBM images in an accurate and high throughput manner. Adhered cells can be identified, and morphological features, such as the size and eccentricity of each individual cell can be extracted. MBM videos enable studying the dynamics of cells on the individual level and allow us to determine kinetic properties like adhesion durations and average velocities (for the case of cells that roll or migrate while on the surface). The individual cell data can be aggregated to produce group statistics, such as distributions of morphological features and dynamic quantities, or mean squared displacements. Importantly, the system and method described herein allows us to identify and analyze the properties of hundreds of thousands of cells. MBM can be generalizable to studies of various diseases, including cancer, blood disorders, thrombosis, inflammatory and autoimmune diseases, as well as provide rich datasets for theoretical modeling of adhesion dynamics.

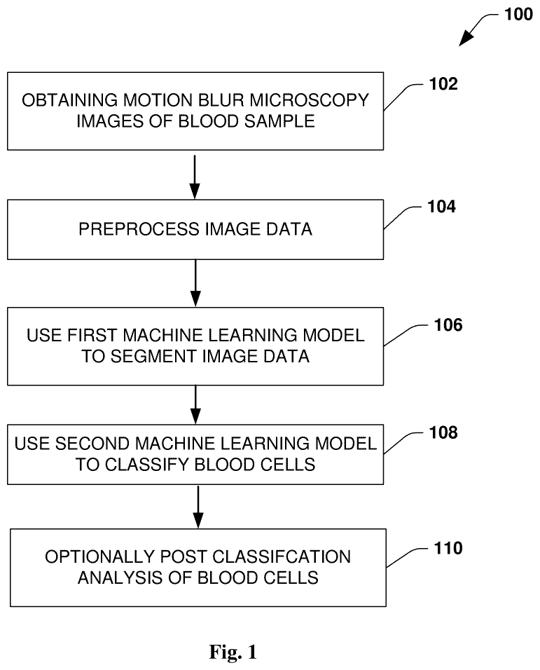

FIG. 1 is a flow diagram depicting an example of a method 100 that may be implemented for classifying blood cells and hemodynamic properties of blood cells. The method can be implemented as program code or modules (e.g., machine readable instructions), which are executable by one or more processors to perform the method 100. The instructions can be stored locally on a computing device that executes the instructions to run the code, or the instructions can be stored and/or executed by remote computing device (e.g., in a computing cloud, web-based or other networked architecture).

The method begins at 102 in which a set of one or more motion blur microscopy (MBM) images of a blood sample flowing through a microchannel of a microfluidic device is obtained. In one example, the microchannel can define one or more cell adhesion regions. Each respective cell adhesion regions can include at least one capturing agent configured to adhere or capture to a cell of interest in a fluid sample when the fluid sample containing the cells is passed through the at least one microchannel.

As a further example, the fluid sample includes undiluted whole blood and the image data thus includes one or more MBM images of the blood sample within an adhesion region of a microchannel of a microfluidic device. Other microfluidic devices can be used in other examples. In an example, the blood sample is expected to include red blood cells (RBCs) and/or white blood cells (WBCs). In some embodiments, the RBCs include sickle RBCs (sRBCs). In other embodiments, the WBCs include T-cell, such as CAR T-cells.

As one example, the microfluidic device can be fabricated by lamination of a polymethylmethacrylate (PMMA) plate, custom laser-cut double-sided adhesive film which has a thickness of about 50 μm (3M, Two Harbors, MN) and an UltraStick adhesion glass slide (e.g., commercially available from VWR International, LLC of Radnor, Pennsylvania). The microchannels can be functionalized with bioaffinity ligands, such as laminin (e.g., available from Sigma-Aldrich of St. Louis, MO). Laminin is a sub-endothelial protein with preferential adherence to sRBCs over healthy RBCs. As a result, using laminin (or another type of similar functionalization agent) to define the adherence region allows the image analysis to focus on sRBC characterization. Other capturing agents can be used depending on the cells of interest. For example, when the fluid sample is synovial fluid, the cells of interest can be white blood cells (WBCs). For demonstrating Jurkat or CAR-T motility, the microchannels can coated with either P- or E-selectin (human, CD62P and CD62E). For microfluidic surface endothelialization, the channels can be incubated human fibronectin (Sigma-Aldrich) and seed with HUVECs.

As a further example, the microfluidic device can be implemented according to the disclosure in any of International Application No. PCT/US2018/022888, filed on 16 Mar. 2018, International Application No. PCT/US2020/058272, filed on October 2020, and International Application No. PCT/US2020/060227 filed 12 Nov. 2020, each of which is incorporated herein by reference.

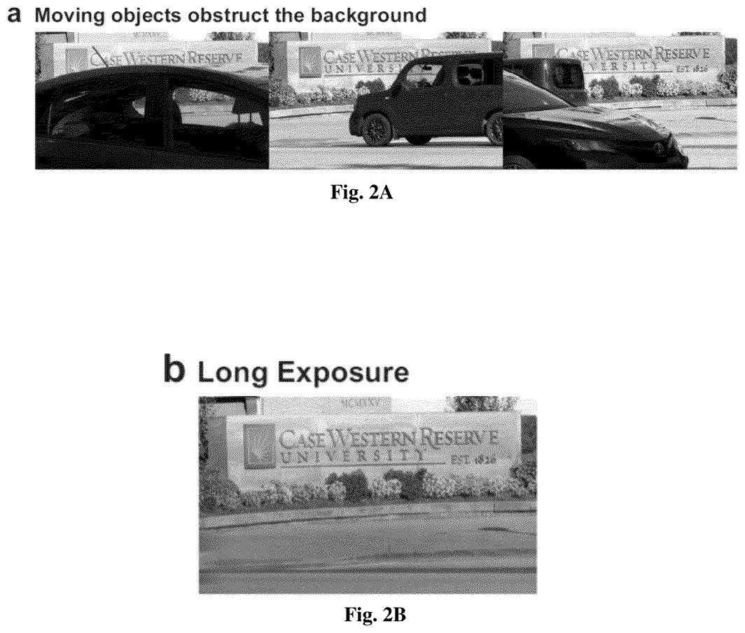

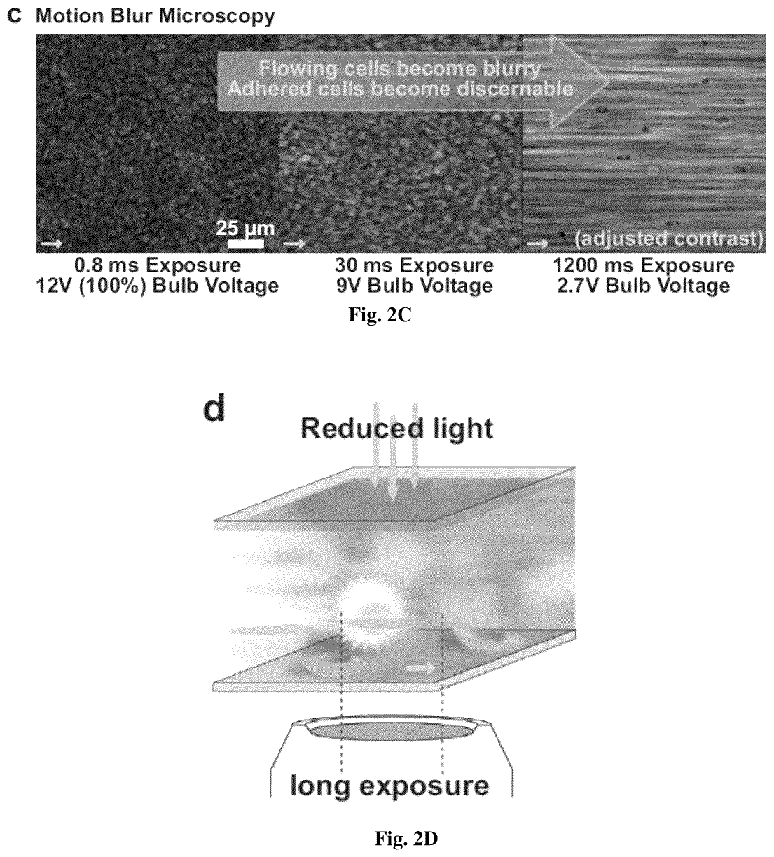

Blood samples are sent through the microchannel to produce either individual MBM images or videos. As illustrated in FIG. 2, MBM works by reducing the light source and increasing the exposure time, resulting in streaks of flowing cells that generate noisy images. Moving objects in the foreground obstruct the stationary objects in the background. By adjusting camera exposure, moving objects are blurred to obtain a clear view of the background. The same principle is applied to microscale whole blood flow. For example, FIG. 2C shows three views of whole blood flow in the same microscopic field with different microscope settings. Increasing the camera exposure (integration time) helps blur the foreground, which consists of non-adhered (flowing) cells. Long exposure results in excess brightness which is then compensated by a reduction of the light source voltage to obtain a clear view of the adhered cells in the background. Yellow arrows show the flow direction. The shear rate of the flow is 50 s⁻¹ which is enough to induce the blur at 1200 ms integration time. FIG. 2D shows a schematic illustration of the microfluidic channel, with clearly visible adhered cells and blurry flowing cells.

By way of example, for MBM imaging, microchannels can be visualized with Olympus CellSens software using an Olympus IX83 inverted microscope and QImaging EXi Blue CCD camera with 10× objective (numerical aperture 0.3, pixel area 6.5 μm²). To induce motion blur, camera exposure can be set to 1.2 s. Images and videos of the microchannel surface can be saved uncompressed to reduce noise. Frame rates of the videos can be kept at (1/1.2) s⁻¹, which is the maximum frame rate for a 1.2 s exposure time. High integration time can be compensated for by adjusting the voltage of the light source to 2.7 V (maximum 12 V).

In one example, unprocessed or leuko depleted whole blood can be loaded into a constant displacement syringe pump (NE-1000, New Era Pump Systems Inc.). MBM with automated analysis requires a minimum flow velocity of about 150 μm/s in the background to create the minimum particle streak for MBM. Higher background flow velocity yields better distinction of cellular interactions. A 50/50 light distribution mode between the camera and the eyepieces can be selected, which allows doubling the exposure time at the same brightness level. The MBM imaging can be performed in a dark room to prevent room lights, or sunlight, from introducing non-uniformity to image lightness. The flow velocity can be kept at about 500 μm/s to 3500 μm/s for demonstrating sRBC adhesion to laminin or CAR-T cell adhesion to E-selectin.

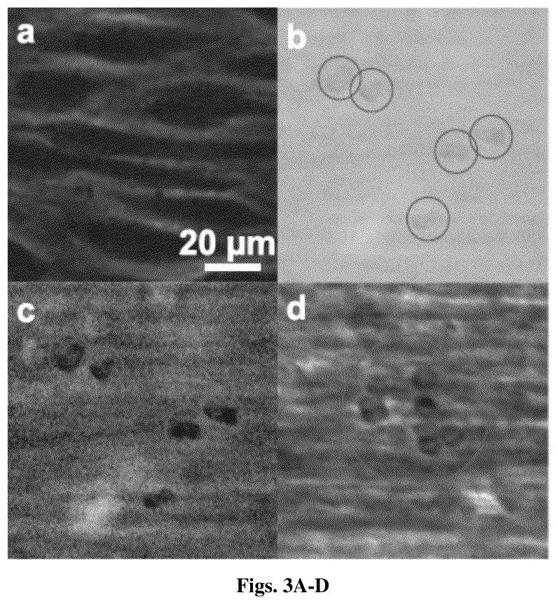

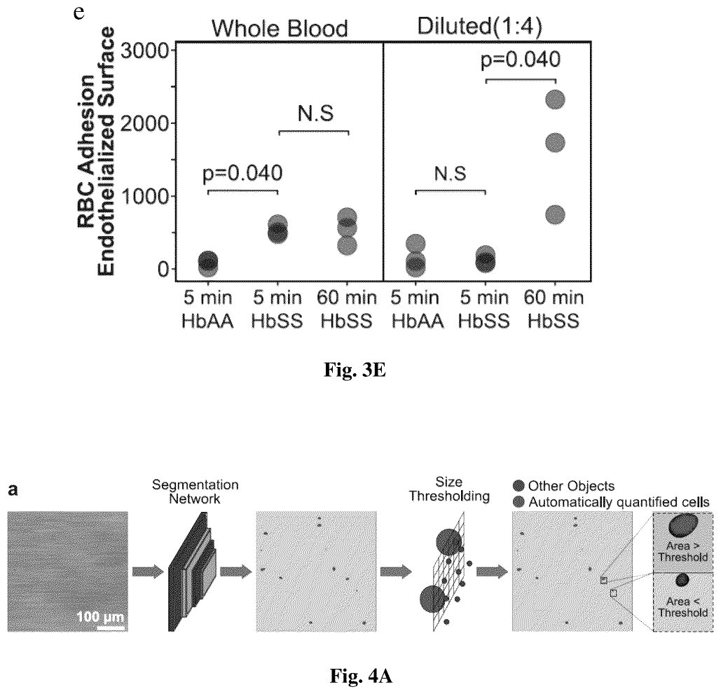

MBM leverages blurring to make the cellular interactions that take place at slower velocity scales discernable. For example, we showcase MBM on protein functionalized surfaces, but MBM also works on endothelialized surfaces. We show that the numbers of adhesive sickle red blood cells (sRBCs) from individuals with sickle cell disease interacting with the endothelial surface are greater than those of healthy RBCs, and diluting the whole blood samples may diminish these interactions or result in aberrant interactions (FIG. 3). Individual cells with a velocity substantially less than the bulk flow (i.e., immobile, adhered cells, or those that are rolling/migrating while in contact with the surface) can be visualized within the whole blood flow. MBM allows in vitro analysis of various static and dynamic properties of cellular interactions, all while mimicking key in vivo conditions.

The obtained set of one or more MBM images or MBM image data can be stored in memory. The MBM image data along with executable instructions may be stored in one or more non-transitory storage media, such as non-volatile data storage (e.g., a hard disk drive, a solid-state drive, flash memory, etc.). It will be appreciated that the storage medium may include a single discrete article or multiple articles interconnected to allow for data transfer among them, for example, via an associated bus or a local or wide-area network connection.

Returning to FIG. 1, the method at step 104 further includes processing the MBM imag data. The image preprocessing can include loading the MBM image data from memory (e.g., stored at 102) which will evenly crop the original whole channel image into smaller tiles and resize respective tiles so that they fit into the input layer for of a neural network model, as disclosed herein. The MBM image data can be provided automatically from a process or it can be selected from memory in response to a user input. The preprocessing at 104 can include cropping the input image (e.g., a large whole channel image) into smaller image tiles. The cropped size can vary depending on the size requirements for the input layer of the machine learning model (e.g., shown at 106).

At 106, the method includes using a first machine learning model to segment image data. For example, the preprocessed input image can be provided to a pre-trained neural network model. For example, the first machine learning model is pre-trained to segment each image tile (e.g., from the respective image tiles provided at 104) by classifying individual pixels and providing respective output images of cells of interest based on respective trained categories that correlate to physical properties of cells, such as cell size. In an example, the machine learning model is configured to detect and distinguish cells adhered to the functionalized channel with endothelial proteins relative to other objects (e.g., other RBCs, WBCs or other non-blood cell objects) provided in the input image tiles. The machine learning model can also output a count to identify a number objects classified in each category based on the segmented respective output images of cells of interest provided at 106.

In the following examples described herein, the first machine learning model is an artificial neural network and, in particular, a convolutional neural network configured to perform semantic segmentation of each input image. In other examples, the machine learning model may one or more of a decision tree, a support vector machine, a clustering process, a Bayesian network, a reinforcement learning model, naïve Bayes classification, a genetic algorithm, a rule-based model, a self-organized map, and an ensemble method, such as a random forest classifier or a gradient boosting decision tree). The training process of a given model will vary with its implementation, but training generally involves a statistical aggregation of training data into one or more parameters associated with the output classes. In some examples, the machine learning model that analyzes the image is described as a first model because the method 100 includes multiple phases of deep learning, in which the first phase includes a machine learning model that implements segmentation and detection of objects that drives another phase of deep learning that includes another machine learning model to implement object classification.

In one example, the machine learning network segments every pixel in an MBM image into one of two categories: adhered or background. Adhered pixels correspond to a pixel of the image belonging to an adhered object, with the remaining pixels falling into the background category. To train the network, one or more MBM images are chosen as training/validation images, and the rest of the images are set aside for testing. A labeled mask of the training/validation images is created, by manually labeling each pixel as adhered or background. Each labeled training/validation mask, as well as each training/validation image, is then split into tiles of size 150×150 pixels, which are subsequently resized to 128×128 pixels, as that is the required input of the segmentation network. Once all of the training/validation tiles are compiled, the segmentation network can be trained for a particular task.

Ideally, the chosen training/validation images are dense in the objects of interest, which will combat any class imbalance during training. Labeled masks for the training/validation images can be created by coloring each pixel of the images one of two colors: one color for adhered pixels, and one color for background pixels. This process can be completed by first coloring all adhered pixels one color using the software GIMP. Any non-colored pixels can then be filled in automatically with a Python script. When resizing tiles from 150×150 pixels to 128×128 pixels, the cubic interpolation method of the Python library OpenCV can be used.

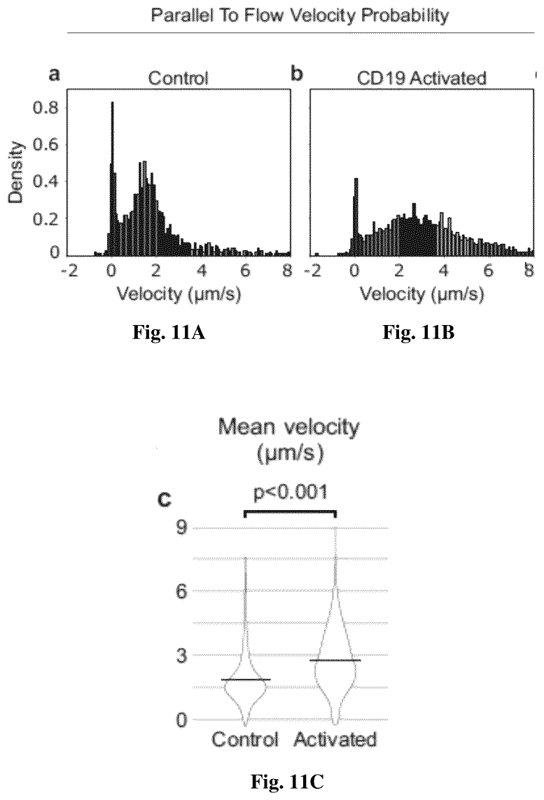

To train the segmentation networks (with architecture summarized in FIG. 12), a custom data generator can be used, which loads tiles into the network in batches of 32 and applies data augmentation. Data augmentations can include rotations of 90, 180, 270, and 360 degrees, horizontal flips, and vertical flips. In total, the augmentations can produce 8 unique orientations of each tile. A stop function can also be included in the training, which automatically stops the training after the validation loss has not decreased for a number of consecutive epochs. The weights of the network after execution of the stop function can be used as the trained network weights.

In some embodiments, the loss function used for optimization can be a linear combination of the categorical cross-entropy loss (Cat) from the Keras library, and the Jaccard loss (Jac), of the form:

$$L = α• Cat + ( 1 - α• ) Jac \qquad ( 1 )$$

where α is a real number in the range [0,1]. The training was completed in both instances using an Adam optimizer. By way of example, the number of epochs before stopping, a value, and learning rate used for training of sRBC adhesion to laminin and CAR-T cell adhesion to E-selectin were [7,0.58,0.007] and [8,0.18,0.009] respectively.

In one example, a first segmentation network was trained on three full MBM image taken from three different sources functionalized with laminin for sickle red blood cell adhesion. The chosen images originally had dimensions of 15171×2391, 15171×4782, and 5057×1196 pixels respectively. When creating the masks for this segmentation network, pixels corresponding to the adhered category were manually colored white. After the manual labeling was completed, the masks were sent through a python code which labeled all background pixels as black. The number of training/validation tiles generated for this segmentation network was 2163 tiles. Augmentations were applied to the training/validation tiles, resulting in 17304 total tiles for training/validation. 70 percent of the tiles were used for training, 20 percent were used for validation during individual training sessions, and 10 percent were used for comparing trained models against one another during hyperparameter optimization. When splitting the tiles into training/validation1/validation2 groups, the training set was drawn from only one of the three image sources, while the validation1/validation2 sets were drawn from the other two image sources. Furthermore, the training/validation1/validation2 datasets were stratified, to make sure that the relative distributions of classes were equal for all datasets.

In another example, a second segmentation network was trained on tiles from 30 frames of MBM videos extracted from three separate sources of CAR-T cell adhesion to E-selectin. Ten frames from each source were used, where the ten frames were chosen from the last 100 frames of the video in increments of ten. Each frame had dimensions of 1392×1040 pixels. When creating the masks for this segmentation network, pixels corresponding to the adhered category were manually colored green. After the manual labeling was completed, the masks were sent through a python code which labeled all background pixels as black. These frames generated a total of 767 training/validation tiles. Augmentations were applied to the training/validation tiles, resulting in a total of 6136 total tiles for training/validation. 70 percent were used for training, 20 percent were used for validation during individual training sessions, and 10 percent were used for comparing trained models against one another during hyperparameter optimization. When splitting the tiles into training/validation1/validation2 groups, the training set was drawn from only one video source, while the validation1/validation2 sets were drawn from the remaining two sources. Furthermore, the training/validation1/validation2 datasets were stratified, to make sure the relative distributions of classes were equal for all datasets.

The output of the segmentation network 106 is a fully segmented image, where each pixel belongs to one of two classes. At 108, the method includes extracting groups of adhered pixels and classifying groups to identify cell types or hemodynamic properties. Generally, the cell identification task falls into one of two cases. In the first case, only one adhered cell type is expected to be distinguishable in the MBM image. In the second case, two or more cell types are expected to be distinguishable in the MBM image.

When only a single cell type of interest is expected to adhere to the functionalized channel, groups of adhered pixels can be classified using a physical property of the group. For example, looking at the attachment of sRBCs to laminin or CAR-T cells to E-selectin, the vast majority of groups of adhered pixels correspond to one cell type. In this case, a size threshold can be used to distinguish adhered cells from groups of adhered pixels falsely classified by the segmentation network as adhered.

When training both segmentation networks, the size threshold for the classification of groups of adhered pixels can be optimized with the objective to produce the closest agreement between human and machine counts of relevant cells. To find the optimum threshold, an array of threshold values can be used to count cells for an array of inputs. The automated cell counts can then be compared to manual cell counts, and the threshold which produces the greatest agreement can be chosen. For example, for single-cell type analysis, two optimum thresholds were found, one for sRBC adhesion to laminin and one for CAR-T cell adhesion to E-selectin.

For example, in the laminin case, 174 MBM images were analyzed using a trained segmentation network, and size thresholds in the range 0-200 px (corresponding to the area of the adhered pixel group), in increments of ten, were used to produce sRBC counts. Agreement between human and machine counts was checked for each threshold value, and the optimum threshold was found to be 90 pixels (38 μm²), where groups of adhered pixels with a size greater than 90 pixels were classified as sRBCs, and groups of adhered pixels with size less than 90 pixels were classified as other. For the CAR-T case, 172 frames from an MBM video were analyzed using a trained segmentation network, and size thresholds in the range 0-200 px, in increments of ten, were used to produce CAR-T cell counts. The optimum threshold was found to be 30 pixels (13 μm²).

Optionally, at step 110, after segmentation of MBM images and classification of the cells are completed, the groups of adhered pixels that are positively classified as the cell type(s) of interest can be further analyzed. Properties like the size, eccentricity, and position of each individual cell are easily obtainable. These static data can be analyzed individually or aggregated together to produce group statistics. When MBM video is available, the dynamics of cells under flow can be analyzed. A parameter for each adhesion event called the survival time can be defined, e.g., the time from when a cell attaches to the surface until it detaches or leaves the frame. With the survival time in hand, as well as the static data for each frame, dynamic data for each cell can be generated. Individual cell dynamics like average velocity, average size, and average eccentricity can be found, or, cells can be aggregated and produce group statistics like mean-squared displacement, or distributions of dynamic quantities.

The computing device implementing the method 100 can include a display or other output device configured to provide a user-perceptible output (e.g., a graphical and/or textual representation) based on the method that is executed. The output can include a quantity (e.g., count) of cells according to their determined classification type or sub-type (e.g., determined at 106 and/or 110). The classification results, including from the segmentation/classification network, can also be included in an output to provide another quantitative or qualitative indication of disease state (e.g., SCD) based on the methods disclosed herein. In a further example, the method 100 and the classification at 108 to successive frames of a video, counting cells of specific types in each video frame. In this way, the behavior of individual cells could be tracked in a series of successive frames. For instance, an adhered cell may attach, stay in a given location for several frames, and then detach. The output, in this example, would be the number of frames that the cell has adhered, which would provide useful information about the adhesion strength under flow conditions for different cell types.

MBM video analysis brings forth additional challenges beyond those of individual images. In particular, for tracking cells over time, it should be determined whether cells in consecutive frames are the same cell or not. Two distinct issues arose when making this determination: duplicate counts, and tracking motile cells.

Duplicate counts occur when the segmentation network erroneously segments a single group of adhered pixels as two distinct groups of adhered pixels, whose edges are close, but not touching. To minimize the duplicate counts, the centroids of any two groups of adhered pixels can be checked to determine if they are within 50 pixels (1 px=0.322 μm) of one another. Also, the shortest distance between the edges of all such pairs can be computed, and if this distance was 5 pixels or fewer for any pair, the two groups of adhered pixels are considered to be one. In this case, the two groups of adhered pixels are replaced by the smallest polygon that contains them, known as the convex hull.

Another case of duplicate counting occurs if the cell classification misclassifies an adhered cell as other for any amount of frames between two positive classifications. This error would cause the analysis to detect a new group of adhered pixels, despite it being the same cell. This can be avoided by giving each adhesion event a disappearance allowance. Each cell for an adhesion event was allowed to disappear for a number of frames before considering the adhesion event completed. If a cell disappeared for a number of frames below the allowance and then reappeared, the adhesion event would continue as if the misclassification never occurred. By way of example, for sRBCs, we allowed cells to go undetected for up to two frames and still count as the same cell. For CAR-T cells, they could go undetected for up to nine frames. These frame thresholds helped us to not preemptively end adhesion events.

Interacting motile cells are constantly adhering, detaching, and persisting in their adhesion to the surface, and as such, the results of analyzing many frames present a complex picture of many adhesion events happening at various times. Thus, we devised rules by which to determine if a cell in consecutive frames was a new adhesion event or a continuation of a prior adhesion event. If a positively identified group of adhered pixels was within 5 pixels in any direction of a positively identified group of adhered pixels in the previous frame, the group of adhered pixels was classified as a continuation of the adhesion event. Otherwise, the positively identified group of adhered pixels can be classified as the beginning of a new adhesion event. Also, if a positively identified group of adhered pixels are within 5 to 30 pixels in the direction of the flow, this group of adhered pixels can be classified as a motile adhesion event. These movement rules plus the frame thresholds protected us from splitting a singular adhesion event into two due to any misclassifications by the automated analysis.

### Example

In this example, we describe a practical, accessible, and easily adaptable microscopy method that enables real-time imaging of dynamics of cellular interactions under whole blood flow in vitro by completely eliminating the need for blood sample dilution. We call our approach motion blur microscopy (MBM). MBM leverages blurring to make the cellular interactions that take place at slower velocity scales discernable (FIG. 2). For simplicity, we showcase MBM on protein functionalized surfaces, but MBM also works on endothelialized surfaces. We show that the numbers of adhesive sickle red blood cells (sRBCs) from individuals with sickle cell disease interacting with the endothelial surface are greater than those of healthy RBCs, and diluting the whole blood samples may diminish these interactions or result in aberrant interactions (FIG. 3). Individual cells with a velocity substantially less than the bulk flow (i.e., immobile, adhered cells, or those that are rolling/migrating while in contact with the surface) can be visualized within the whole blood flow. MBM allows in vitro analysis of various static and dynamic properties of cellular interactions, all while mimicking key in vivo conditions.

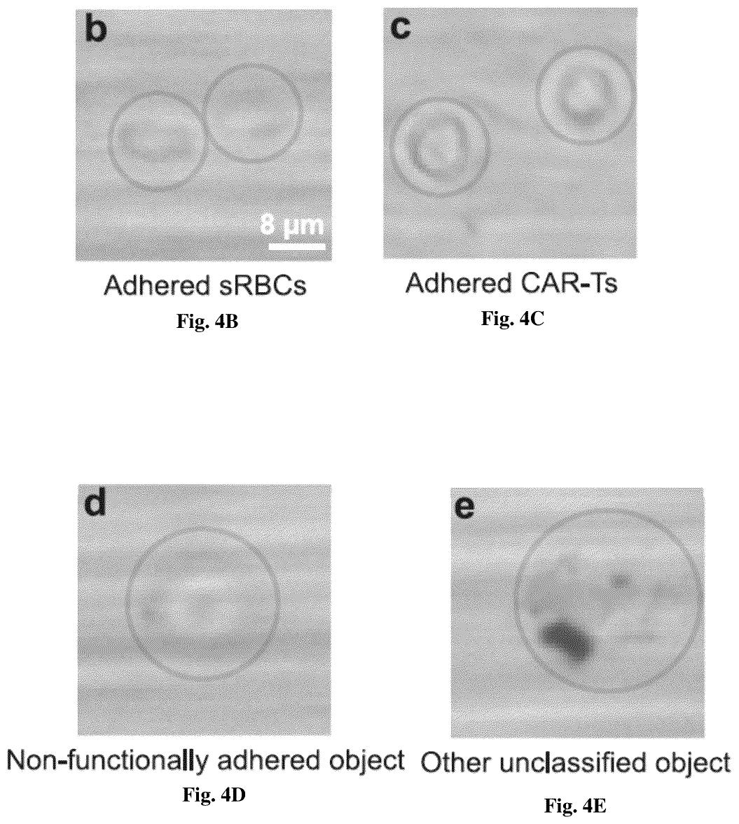

MBM works by reducing the light source and increasing the exposure time, resulting in streaks of flowing cells that generate noisy images. Thus, to identify and analyze adhered cells, an experimenter must spend a considerable amount of time and effort to distinguish cells. While this may not be a difficult task when analyzing individual MBM images, manually analyzing dynamic interactions from MBM videos consisting of hundreds or thousands of frames can be impractical and error-prone. Therefore, we developed an automated machine-learning-based analysis, which can efficiently characterize the dynamics of cellular interactions in MBM videos. The automated analysis is a two-phase system, where phase one identifies groups of pixels in an MBM image corresponding to adhered cells, and phase two classifies these groups by cell type (FIG. 4A). The phase one task is completed using a machine learning segmentation network. The phase two task is completed by classifying cells by their size, or by using a machine learning classification network, depending on the complexity of the system under study. FIGS. 4(B-E) shows examples of adhered regions that one might expect the automated analysis to classify. FIGS. 4B, C corresponds to regions of interest, containing sRBCs and chimeric antigen receptor T-cell (CAR-T) cells, respectively, and the automated analysis should correctly identify these regions as adhered cells. FIG. 4D depicts a typical object adhered to the surface that is not protein functionalized, and FIG. 4E shows other stationary objects that are not cells. The automated analysis pipeline distinguishes these non-functionally adhered objects and other non-cell objects from the adhered cells of interest.

With our automated analysis, we can process MBM images in an accurate and high throughput manner. Adhered cells can be identified, and morphological features, such as the size and eccentricity of each individual cell can be extracted. MBM videos enable studying the dynamics of cells on the individual level and allow us to determine kinetic properties like adhesion durations and average velocities (for the case of cells that roll or migrate while on the surface). The individual cell data can be aggregated to produce group statistics, such as distributions of morphological features and dynamic quantities, or mean squared displacements. Importantly, the method allows us to identify and analyze the properties of hundreds of thousands of cells. These data can be used to help us understand cellular adhesion dynamics, or be used in clinical studies. Since our approach relies on a basic experimental microscopy setup, we anticipate that it will be highly accessible to the broader research community.

### Methods

### Blood Sample Collection and Microfluidic Adhesion Assays

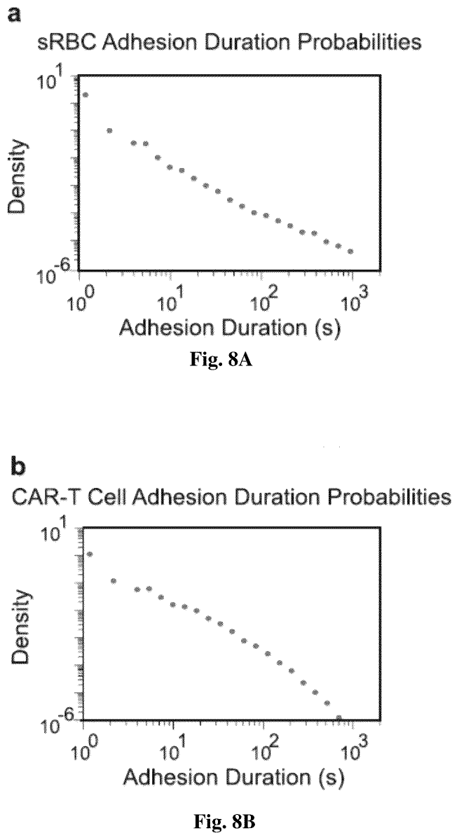

All research complies with the ethical regulations, approved by University Hospitals Institutional Review Board office (#05-14-07C). Surplus EDTA-anticoagulated whole blood samples were collected with the informed consent of the subjects under a protocol registered at www.clinicaltrials.gov as NCT02824471, “Sickle Cell Disease Biofluid Chip Technology”. The microfluidic platforms were fabricated by lamination of 50 μm laser-cut double-sided adhesive film between APTES coated glass slide and 3.2 mm thick Poly(methyl methacrylate) (PMMA) cover. Protein functionalization of the microchannels was achieved by injecting the channels with N-g-Maleimidobutyryloxy succinimide ester (0.28% vol/vol) followed by incubation with desired protein for 1.5 h at room temperature. For studying the adhesion of HbAA-containing red blood cells (healthy control with normal hemoglobin), unprocessed whole blood samples from healthy donors with no known hemoglobinopathies were used. For studying the adhesion of sRBCs, unprocessed whole blood samples from subjects with homozygous sickle cell disease (HbSS) were used and microchannels were coated with laminin (murine, laminin-1). For demonstrating Jurkat or CAR-T motility, the microchannels were coated with either P- or E-selectin (human, CD62P and CD62E). For microfluidic surface endothelialization, the channels were incubated overnight at 4° C. with 10% human fibronectin (Sigma-Aldrich). HUVECs (Lonza) were then seeded into each channel at a concentration of about 10 million cells/μL and channels were cultured under flow conditions created by a peristaltic pump for 2-3 days until confluent. Whole blood samples from subjects with no hemoglobinopathies were first leuko-depleted and then premixed with Jurkat or CAR-T cell populations at 40% hematocrit in Hank's buffer containing calcium to retain the leukocyte activity by replenishing EDTA-depleted Ca++ in blood samples (FIG. 8). HbSS whole blood was diluted 1:4 with PBS for the dilution experiment. Hypoxic conditions (SpO₂ of ˜83% in the blood sample), were achieved using an in-house developed micro-tube gas exchanger. Highly gas permeable inlet tubing was fed through an impermeable tube which was connected to a 4 psi 95/5% N₂/CO₂ mixture source to allow parallel-flow gas exchange. To prevent non-specific cellular interactions, microchannels were incubated with bovine serum albumin at 4° C. overnight, and additionally, 250 μl of bovine serum albumin (20 mg/mL) was injected into the microchannels at room temperature with a 5 l/min flow rate 2 h prior to the experiments.

### CAR T-Cell Culture

CAR T-cells were obtained from the Hematopoietic Biorepository and Cellular Therapy core at Case Western Reserve University. These cells manufactured according to ethics and guidelines from University Hospitals Cleveland Medical Center (UHCMC IRB #03-18-01C). Briefly, CD3+ T cells were collected from whole blood samples using the Miltenyi CD3 T cell isolation kit (North Rhine-Westphalia, Germany) and transduced with a CD19-directed CAR vector. Cells were cultured in TexMACS media with IL-7 and IL-15 (Miltenyi Biotec; North Rhine-Westphalia, Germany). Cells were then cryopreserved until experimentation. Upon thawing patient samples, cells were cultured in RPMI 1640 medium with 10% fetal bovine serum, 100 μ/mL pen/strep (Thermo Fisher, Waltham, MA, USA), 2 mM glutamax (Thermo Fisher, Waltham, MA, USA). For CD19 activation, CAR-T cells were added to a solution of IL-2 culture media containing CD19+RAJI cells obtained from American Type Culture Collection (Manassas, VA, USA).

### Motion Blur Microscopy

Microchannels were visualized with Olympus CellSens software using an Olympus IX83 inverted microscope and QImaging EXi Blue CCD camera with 10× objective (numerical aperture 0.3, pixel area 6.5 μm²). To induce motion blur, camera exposure was set to 1.2 s. Images and videos of the microchannel surface were saved uncompressed to reduce noise. Frame rates of the videos were kept at (1/1.2) s⁻¹, which is the maximum frame rate for a 1.2 s exposure time. High integration time was compensated for by adjusting the voltage of the light source to 2.7 V (maximum 12 V). It should be noted that it is key to adjust the focus map for the surface of interest before the microchannels are injected with the sample. Unprocessed or leuko depleted whole blood was withdrawn into a syringe which then was loaded into a constant displacement syringe pump (NE-1000, New Era Pump Systems Inc.). MBM with automated analysis requires a minimum flow velocity of ˜150 μm/s in the background to create the minimum particle streak for MBM. Higher background flow velocity yields better distinction of cellular interactions. 50/50 light distribution mode between the camera and the eyepieces was selected, which allows doubling the exposure time at the same brightness level. We performed all the experiments in a dark room. This is important because MBM is a low-light technique and room lights, or sunlight, may introduce non-uniformity to image lightness. The flow velocity was kept at ˜500 μm/s for 20 min for demonstrating sRBC adhesion to laminin. For the demonstration of CAR-T cell adhesion to E-selectin, the flow velocity was swept linearly from 500 μm/s to 3500 μm/s in 10 min.

### Automated Image Analysis

To analyze MBM images, we developed an automated analysis procedure with two distinct phases. Phase 1 performs segmentation on MBM images at the pixel level and phase 2 classifies groups of pixels by cell type. After classification, we can perform further analysis on the classified cells (i.e., morphology characterization and trajectory tracking). While we focus on three experimental examples, our approach can easily be generalized to other systems.

### Phase 1: Pixel Segmentation

Phase 1 uses a machine learning network to segment every pixel in an MBM image into one of two categories: adhered or background. Adhered pixels correspond to a pixel of the image belonging to an adhered object, with the remaining pixels falling into the background category.

The segmentation network is based on a modified U-Net architecture. To train the network, one or more MBM images are chosen as training/validation images, and the rest of the images are set aside for testing. A labeled mask of the training/validation images is created, by manually labeling each pixel as adhered or background. Each labeled training/validation mask, as well as each training/validation image, is then split into tiles of size 150×150 pixels, which are subsequently resized to 128×128 pixels, as that is the required input of the seg-mentation network. Once all of the training/validation tiles are com-piled, we can then train the segmentation network for our particular task.

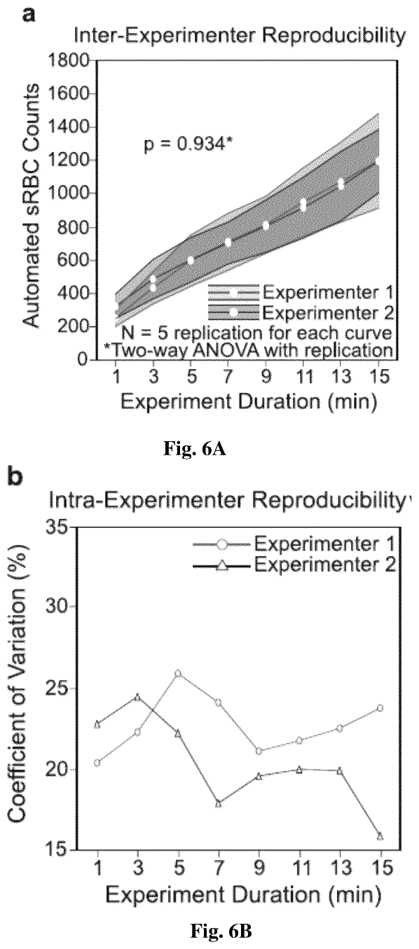

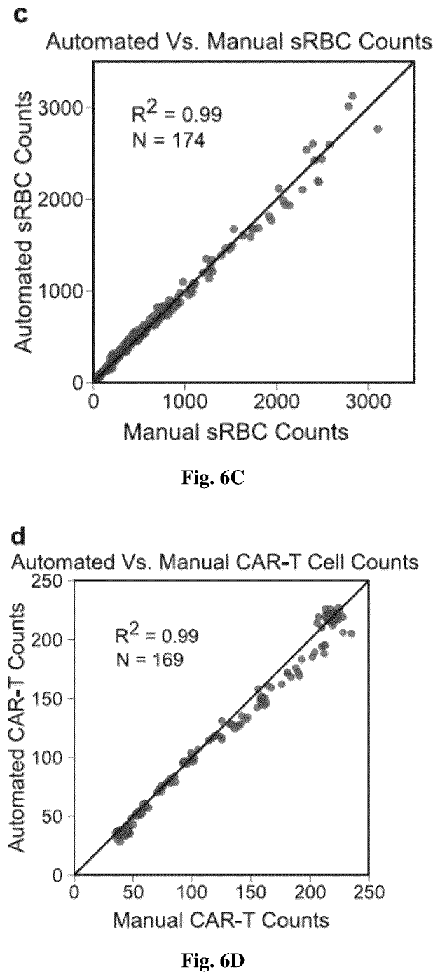

We trained and used two distinct segmentation networks, one for analysis of sRBC adhesion to laminin, and one for analysis of CAR-T cell adhesion to E-selectin. The first network was trained/validated on tiles from a subset of 3 out of 177 MBM images. The training tiles are drawn from one of the three images, while the validation tiles are drawn from the two other images. The remaining 174 images were set aside for testing the network's ability to count adhered sRBCs (FIG. 6C). In total, there were 2163 tiles used for training/validation. The second network was trained on tiles from the frames of three MBM video sources. For each video source, ten evenly spaced frames were extracted from the final 100 frames, giving 30 frames in total for training/validation. The training tiles are drawn from one of the video sources, while the validation tiles are drawn from the two other video sources. 169 of the remaining frames from one of the video sources were set aside for testing the network's ability to count adhered CAR-T cells (FIG. 6D). In total, there were 767 tiles used for training/validation. In both cases we implemented a stratified split of 70%/20%/10% training/validation1/validation2, where validation set 1 was used for validating during individual training sessions, and validation set 2 was used for comparing trained models against one another for the purposes of hyperparameter optimization.

Ideally, the chosen training/validation images are dense in the objects of interest, which will combat any class imbalance during training. Labeled masks for the training/validation images were created by coloring each pixel of the images one of two colors: one color for adhered pixels, and one color for background pixels. This process was completed by first coloring all adhered pixels one color using the software GIMP. Any non-colored pixels were then filled in automatically with a Python script. When resizing tiles from 150×150 pixels to 128×128 pixels, the cubic interpolation method of the Python library OpenCV was used.

To train the segmentation networks (with architecture summarized in FIG. 12), a custom data generator was created, which loaded tiles into the net-work in batches of 32 and applied data augmentation. Data augmentations included are rotations of 90, 180, 270, and 360 degrees, horizontal flips, and vertical flips. In total, the augmentations can produce 8 unique orientations of each tile. A stop function was included in the training, which automatically stopped the training after the validation loss had not decreased for a number of consecutive epochs. The weights of the network after the stop function was executed were used as the trained network weights. The loss function used for optimization was a linear combination of the categorical cross-entropy loss (Cat) from the Keras library, and the Jaccard loss (Jac), of the form:

$$L = α• Cat + ( 1 - α• ) Jac \qquad ( 1 )$$

where α is a real number in the range [0,1]. The training was completed in both instances using an Adam optimizer. The number of epochs before stopping, a value, and learning rate used for training of sRBC adhesion to laminin and CAR-T cell adhesion to E-selectin were [7,0.58,0.007] and [8,0.18,0.009] respectively. The training was carried out in a Jupyter notebook using an NVIDIA GeForce RTX 2060 SUPER GPU with 8 GB of memory and an AMD Ryzen 7 3700X 8-Core CPU.

The first segmentation network was trained on three full MBM image taken from three different sources functionalized with laminin for sickle red blood cell adhesion. The chosen images originally had dimensions of 15171×2391, 15171×4782, and 5057×1196 pixels respectively. When creating the masks for this segmentation network, pixels corresponding to the adhered category were manually colored white. After the manual labeling was completed, the masks were sent through a python code which labeled all background pixels as black. The number of training/validation tiles generated for this segmentation network was 2163 tiles. Augmentations were applied to the training/validation tiles, resulting in 17304 total tiles for training/validation. 70 percent of the tiles were used for training, 20 percent were used for validation during individual training sessions, and 10 percent were used for comparing trained models against one another during hyperparameter optimization. When splitting the tiles into training/validation1/validation2 groups, the training set was drawn from only one of the three image sources, while the validation1/validation2 sets were drawn from the other two image sources. Furthermore, the training/validation1/validation2 datasets were stratified, to make sure that the relative distributions of classes were equal for all datasets.

The second segmentation network was trained on tiles from 30 frames of MBM videos extracted from three separate sources of CAR-T cell adhesion to E-selectin. Ten frames from each source were used, where the ten frames were chosen from the last 100 frames of the video in increments of ten. Each frame had dimensions of 1392×1040 pixels. When creating the masks for this segmentation network, pixels corresponding to the adhered category were manually colored green. After the manual labeling was completed, the masks were sent through a python code which labeled all back-ground pixels as black. These frames generated a total of 767 training/validation tiles. Augmentations were applied to the training/validation tiles, resulting in a total of 6136 total tiles for training/validation. 70 per-cent were used for training, 20 percent were used for validation during individual training sessions, and 10 percent were used for comparing trained models against one another during hyperparameter optimization. When splitting the tiles into training/validation1/validation2 groups, the training set was drawn from only one video source, while the validation1/validation2 sets were drawn from the remaining two sources. Furthermore, the training/validation1/validation2 datasets were stratified, to make sure the relative distributions of classes were equal for all datasets.

### Phase 2: Cell Classification

The output of the segmentation network is a fully segmented image, where each pixel belongs to one of two classes. In the second phase of the analysis, groups of adhered pixels are extracted and classified. Generally speaking, the cell identification task falls into one of two cases. In the first case, only one adhered cell type is expected to be distinguishable in the MBM image. In the second case, two or more cell types are expected to be distinguishable in the MBM image.

When only a single cell type of interest is expected to adhere to the functionalized channel, we can classify groups of adhered pixels using a physical property of the group. For example, when we look at the attachment of sRBCs to laminin or CAR-T cells to E-selectin, the vast majority of groups of adhered pixels correspond to one cell type. In this case, we can use a size threshold to distinguish adhered cells from groups of adhered pixels falsely classified by the segmentation network as adhered.

When training both segmentation networks, the size threshold for the classification of groups of adhered pixels was optimized with the objective to produce the closest agreement between human and machine counts of relevant cells. To find the optimum threshold, an array of threshold values can be used to count cells for an array of inputs. The automated cell counts can then be compared to manual cell counts, and the threshold which produces the greatest agreement can be chosen. For single-cell type analysis, two optimum thresholds were found, one for sRBC adhesion to laminin and one for CAR-T cell adhesion to E-selectin.

In the laminin case, 174 MBM images were analyzed using a trained segmentation network, and size thresholds in the range 0-200 px (corresponding to the area of the adhered pixel group), in increments of ten, were used to produce sRBC counts. Agreement between human and machine counts was checked for each threshold value, and the optimum threshold was found to be 90 pixels (38 μm²), where groups of adhered pixels with a size greater than 90 pixels were classified as sRBCs, and groups of adhered pixels with size less than 90 pixels were classified as other. For the CAR-T case, 172 frames from an MBM video were analyzed using a trained segmentation network, and size thresholds in the range 0-200 px, in increments of ten, were used to produce CAR-T cell counts. The optimum threshold was found to be 30 pixels (13 μm²).

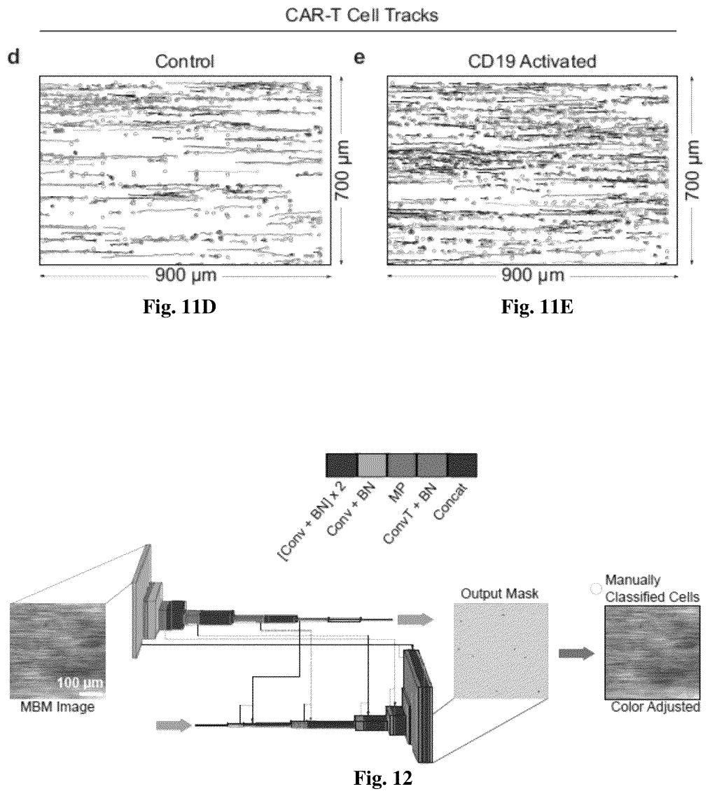

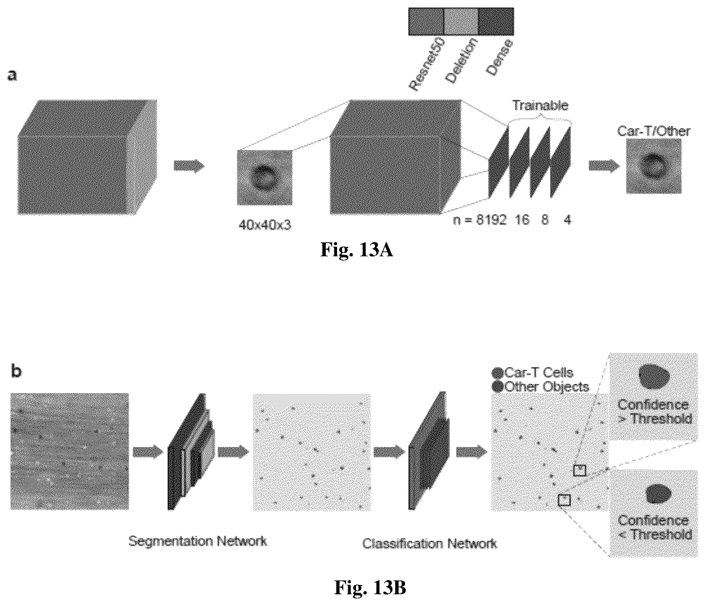

The classification network is a ResNet50 architecture pre-trained on ImageNet³ with additional trainable layers added at the end (FIG. 13). Note there is a special pre-processing applied to MBM images for CAR-T cell adhesion to P-selectin. Each pixel from each MBM image is color adjusted according to the equation:

$${c}_{i}^{′} = \frac{{c}_{i} - ( μ - 3 σ )}{( μ + 3 σ ) - ( μ - 3 σ )} , \qquad ( 2 )$$

where C′ is the color of a pixel after adjustment, Cᵢ is the original color of the pixel, μ is the mean color of the MBM image, and cr is the standard deviation of the color of the MBM image. This color adjustment is not strictly necessary, but it helps the phase 1 segmentation network generalize to a new experimental set-up (the alternative would have been to re-train the phase 1 network for this experimental case).

To train the classification network, 40×40 pixel regions are extracted from MBM images. These regions are centered on groups of adhered pixels classified by the phase 1 segmentation network which have areas larger than the size threshold of 180 pixels. To create a training/validation dataset these regions are manually classified as CAR-T or other. We extracted 8830 regions, and classified 1143 CAR-T cells, and 7687 other, where 70% of regions were used for training, 20% of images were used for validation, and 10% of regions were used for generating F₁ scores and confusion matrices. Data augmentations such as horizontal and vertical flips were applied to the training/validation data set, resulting in four unique orientations for each image, and giving 3200/21523 CAR-T/other images for training and 914/6149 CAR-T/other images for validation. Class weighting was used for all phase 2 training according to the ratios of the classes relative to the total number of im-ages. A stop function was included in the training, which automatically stopped the training after the validation loss had not decreased for 6 straight epochs. The best weights of the network after the 6 straight epochs were used as the trained network weights. The training was carried out via an Adam optimizer with a learning rate of 0.00035 and batch size of 32. The loss function used for optimization was a categorical cross-entropy function from the Keras library. The training was completed on a Jupyter notebook using an NVIDIA GeForce RTX 2060 SUPER GPU with 8 GB of memory and an AMD Ryzen 7 3700X 8-Core CPU.

MBM with Automated Analysis with Multiple Cell Types

When multiple cell types are expected to be visible in an MBM image, phase 2 classification becomes more complicated. In general for this case using a simple physical parameter (like cell size/eccentricity) for groups of adhered pixels will not be sufficient for accurate classification. The reason for this is because the distribution of physical parameters can overlap between the different types. Thus, trying to classify with only a physical parameter may lead to errors. One solution to this problem is using a machine learning classification network for phase 2, which we demonstrate here as a proof of concept to classify CAR-T cell adhesion to P-selectin in the presence of red blood cells (where both CAR-T and red blood cells are expected to adhere).

For simplicity, we re-used the phase 1 segmentation network from analysis of sickle red blood cell adhesion to laminin for analysis of CAR-T cell adhesion to P-selectin. To demonstrate the necessity of a machine learning approach for phase 2, we show simple physical characteristics (eccentricity versus size) of the adhered pixel groups in FIG. 13. As in main text FIG. 4, there are only two distinct peaks: one at small areas (misclassified pixels/detritus) and one at large areas (all adhered cells). The two types of adhered cells do not form distinguishable peaks.

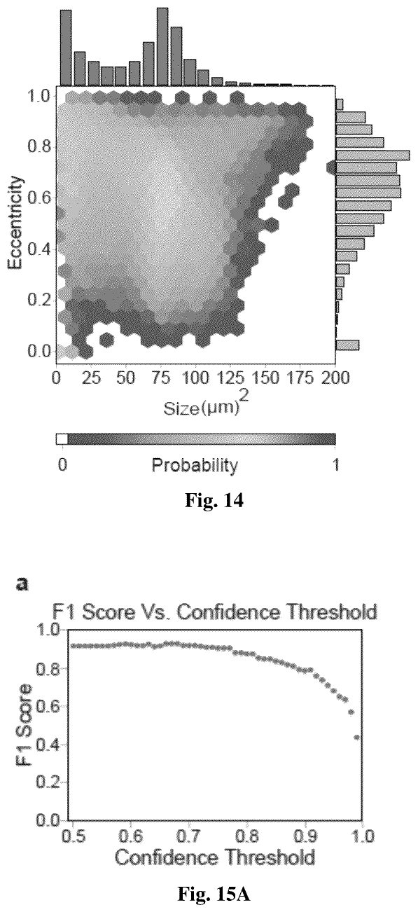

To get reliable CAR-T cell classification, we trained a ResNet50 convolutional neural network. The goal was to identify adhered pixel groups as either CAR-T cells or as non-CAR-T (“other”), starting with pixel groups that had sufficiently large areas to be cells (i.e., after passing the size threshold). The output of this binary classification network is a scalar between 0-1, which can be roughly thought of as the probability of the group being a CAR-T cell. To positively assign a CAR-T classification, we have a choice of confidence threshold, with all network outputs above the threshold labeled as CAR-T. The default threshold would be 0.5, but higher thresholds can also be used to get more conservative assignments. The effectiveness of the binary classification can be measured via an F₁ score, which is the harmonic mean of precision and recall (with values in the range 0-1, where 1 corresponds to perfect classification). As shown in FIG. 14A, most confidence thresholds in the range 0.5-0.99 (except the highest ones) give excellent F₁ scores about greater than 90% for classifying a set of 115/770 CAR-T/other cells. We chose a threshold of 0.67, and for this value, FIG. 15B shows the corresponding confusion matrix and FIG. 15C shows a comparison to manual counting of CAR-T cells by human experts. The excellent agreement (R²=0.95) shows that is possible to have accurate phase 2 classification of M BM images via neural networks even in the presence of multiple cell types.

### Post-Classification Analysis

After phase 1 and phase 2 are completed, the groups of adhered pixels that are positively classified as the cell type(s) of interest can be further analyzed. Properties like the size, eccentricity, and position of each individual cell are easily obtainable. These static data can be analyzed individually or aggregated together to produce group statistics. When MBM video is available, we can analyze the dynamics of cells under flow. We define a parameter for each adhesion event called the survival time the time from when a cell attaches to the surface until it detaches or leaves the frame. With the survival time in hand, as well as the static data for each frame, we can generate dynamic data for each cell. Individual cell dynamics like average velocity, average size, and average eccentricity can be found, or, we can aggregate cells and produce group statistics like mean-squared displacement, or distributions of dynamic quantities.

Identification rules for tracking moving cells in MBM videos. MBM video analysis brings forth additional challenges beyond those of individual images. In particular, we need to track cells over time, requiring us to determine if cells in consecutive frames are the same cell or not. Two distinct issues arose when making this determination: duplicate counts, and tracking motile cells.

Duplicate counts occur when the segmentation network erroneously segments a single group of adhered pixels as two distinct groups of adhered pixels, whose edges are close, but not touching. To minimize the duplicate counts, we checked to see if the centroids of any two groups of adhered pixels were within 50 pixels (1 px=0.322 μm) of one another. Also, the shortest distance between the edges of all such pairs was computed, and if this distance was 5 pixels or fewer for any pair, the two groups of adhered pixels were considered to be one. In this case, the two groups of adhered pixels were replaced by the smallest polygon that contains them, known as the convex hull. One can imagine this shape as the result of stretching a rubber band over the two groups of adhered pixels and letting it rest taut.

Another case of duplicate counting occurs if phase 2 misclassified an adhered cell as other for any amount of frames between two positive classifications. This error would cause the analysis to detect a new group of adhered pixels, despite it being the same cell. We avoid this by giving each adhesion event a disappearance allowance. Each cell for an adhesion event was allowed to disappear for a number of frames before considering the adhesion event completed. If a cell disappeared for a number of frames below the allowance and then reappeared, the adhesion event would continue as if the misclassification never occurred. For sRBCs, we allowed cells to go undetected for up to two frames and still count as the same cell. For CAR-T cells, they could go undetected for up to nine frames. These frame thresholds helped us to not preemptively end adhesion events.

Interacting motile cells are constantly adhering, detaching, and persisting in their adhesion to the surface, and as such, the results of analyzing many frames present a complex picture of many adhesion events happening at various times. Thus, we devised rules by which to determine if a cell in consecutive frames was a new adhesion event or a continuation of a prior adhesion event. If a positively identified group of adhered pixels was within 5 pixels in any direction of a positively identified group of adhered pixels in the previous frame, the group of adhered pixels was classified as a continuation of the adhesion event. Otherwise, the positively identified group of adhered pixels was classified as the beginning of a new adhesion event. Also, if a positively identified group of adhered pixels was within 5 to 30 pixels in the direction of the flow, this group of adhered pixels was classified as a motile adhesion event. These movement rules plus the frame thresholds protected us from splitting a singular adhesion event into two due to any misclassifications by the automated analysis.

### Hyperparameter Optimization of Machine Learning Training

Using the Hyperopt Python package (based on the Tree-Parzen search algorithm), we were able to optimize the hyperparameters for both phase 1 and phase 2 of the ML pipeline. When training phase 1 segmentation networks, we focused on three hyperparameters: the learning rate, the patience (how many epochs we wait without improvement to stop training), and the α parameter from our loss function. For phase two training, we also had three hyperparameters: the learning rate, the patience, and the model architecture. To optimize the hyperparameters in phase 1 and phase 2, the networks were repeatedly trained, while the Hyperopt package suggested parameters in the space. The spaces defined were; [0.0001-0.01] for the learning rate, [0-10] for the patience, [0,1] for the α parameter of phase 1 training, and [VGG16 vs. ResNet50 vs. EfficientNetB3] for the model architecture for phase 2 training. The networks were optimized on the objective of minimizing the Jaccard-Loss on the validation 2 datasets. Once the Jaccard-Loss did not decrease for ten straight parameter samplings, and after at least 50 samplings were made, the optimization was stopped.

### Results

Characterizing fundamental cellular interactions on the single-cell level can reveal small sub-populations of cells that initiate pathogenesis. Here, we focus on two diseases where the cellular interactions in the vascular space carry central importance. Analyzing these subpopulations typically requires intravital microscopy, and thus, serves as a means to demonstrate the effectiveness of MBM in challenging applications. The first of these diseases is sickle cell disease, where hemoglobin, the fundamental protein underlying red blood cells' oxygen transport, polymerizes in a deoxygenated environment. When the hemoglobin polymerizes, a cascade of events can occur, leading to a debilitating disease complication known as a vaso-occlusive crisis. Red blood cells containing polymerized sickle hemoglobin experience physical and chemical changes, resulting in abnormally stiff, dense, and adhesive cells. The second disease in focus is malignant solid tumors where T-cell migration is crucial for immunotherapy of this type of malignancy. Many CAR-T therapies are under development for solid tumors, and they hold great promise for treating refractory cancers. Understanding the migration of T cells, specifically CAR-T cells, would lead to better design of future cell therapies when diapedesis/migration is an important aspect of the therapy, as in the case of solid tumors. An accessible, reliable, single-cell level in vitro method for analyzing CAR-T cell behavior, like MBM, would accelerate over-coming this bottleneck.

To demonstrate MBM and put its validity to the test, three distinct experimental setups were used. One experimental setup used a laminin-functionalized channel to observe sRBCs under flow. A second experimental set-up used an E-selectin functionalized channel to observe CAR-T cells under flow. The final experimental set-up used a P-selectin functionalized channel to observe a combination of red blood cells and CAR-T cells under flow. In this work, we aim to accomplish three main goals. One, we show that MBM with automated analysis is accurate, insofar as it can correctly classify cells for a variety of inputs in a reproducible manner. Two, we show that relevant physical properties of identified cells can be determined. Three, we show that the identified cells and their properties constitute a data set with practical uses, ranging from studying cellular adhesion mechanics to aiding in clinical studies.

Overview of MBM with Automated Analysis

Here we summarize the complete process for analyzing blood samples using MBM. Given a cell type (or types) of interest, we functionalize a microchannel with suitable adhesion proteins. Blood samples sent through the microchannels are then imaged to produce either individual MBM images or videos. Cells of interest adhere to the functionalized protein surfaces and become visible against the blurred foreground of non-adhered flowing cells. The MBM images are analyzed via a two-stage automated process schematically illustrated in FIG. 4A. The first phase consists of a segmentation neural network, which labels pixels from MBM images corresponding to adhered objects, and groups together neighboring labeled pixels. In phase two of the analysis, groups of labeled pixels are then classified by cell type using either a size threshold (in applications where size is a sufficient criterion) or a specially trained classification neural network (in more complicated scenarios). After all of the cells in an MBM image or MBM video have been identified, we then generate data for each identified cell. Morphological properties like the size and eccentricity of each cell can be extracted, as well as dynamical properties such as adhesion duration or mean velocity. Using the automated analysis, we were able to extract properties of individual cells for sRBC/laminin images and videos, a CAR-T/P-selectin video, and CAR-T/E-selectin videos, all together containing hundreds of thousands of adhesion events.

### MBM-Based Cell Classification is Reproducible and Accurate

In cases where only a single cell type is expected to be visible in an MBM image, we demonstrate that we can distinguish groups of adhered pixels identified by the phase one segmentation network from debris and other artifacts using morphological properties of the group.

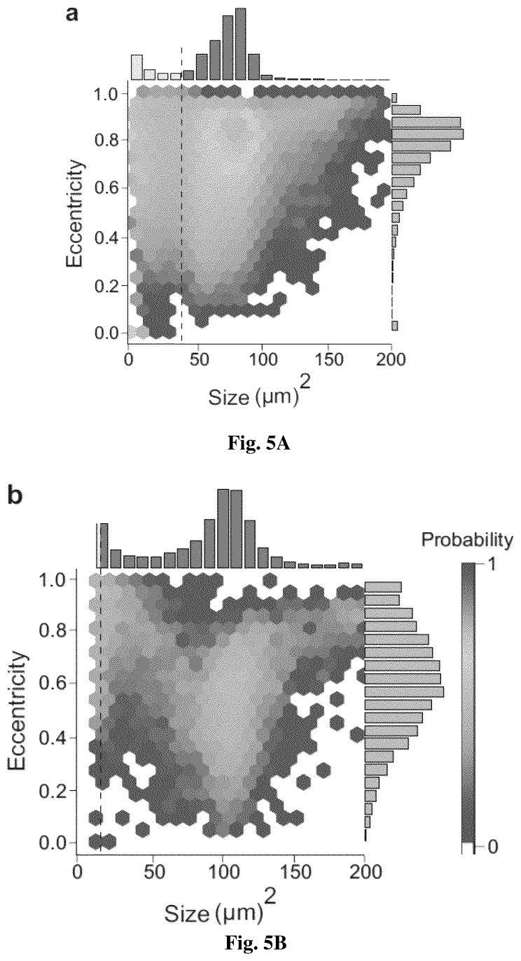

In FIG. 5, we show joint probability densities of the size and eccentricity of all groups of adhered pixels identified by the segmentation network for two collections of MBM images: sRBC adhesion to laminin (FIG. 5A) and CAR-T adhesion to E-selectin (FIG. 5B). The histograms on the top and right edges of the figures are marginal distributions of either the size or eccentricity alone. The joint probabilities reveal three distinct classes of objects: small size/high eccentricity, small size/zero eccentricity, and large size/high eccentricity. The last category corresponds to sRBC or CAR-T cells, and hence we can use a size threshold (indicated by dashed vertical lines) to distinguish cells from other adhered objects (i.e., debris). Each group of pixels above the threshold is classified as a cell.

To validate the inter-experimenter reproducibility of this classification scheme, two researchers replicated five consecutive experiments each. The experimenters used a single tube of blood collected from a homozygous sickle-cell disease subject and analyzed the number of sRBCs using the MBM approach five times, each over a fifteen minute period per experiment. The results did not show a significant difference between the experimenters (FIG. 5A, p=0.934, two-way ANOVA with replication). Furthermore, the coefficient of variation within the replications of each experimenter was <25% for all but one data point (FIG. 6B), an important precision benchmark for bioanalytical method validation. Collectively, these results provide reasonable assurance for the acquisition of meaningful results by showing that our experimental procedure and analysis pipeline are precise and independent of the experimenter.

Finally, we tested the accuracy of the procedure in counting cells, by comparing manual human and automated counts of adhered cells in various MBM images for both sRBC adhesion to laminin (FIG. 6C, N=174) and CAR-T adhesion to E-selectin (FIG. 6D, N=169). The pipe-line performed very well, with an R² value of 0.99 for both the sRBC and CAR-T cases. Importantly, the counts remained accurate even as the number of adhered cells becomes large in an individual image.

MBM can provide high-throughput single-cell dynamic data The effectiveness of MBM at analyzing adhered cells in static images generalizes to videos, allowing us to characterize the dynamics of adhered cells on protein-functionalized surfaces. To facilitate this, we combined the classification procedure described above (applied to each frame of the video) with a cell tracking algorithm. The tracking analysis distinguishes the motion of adhered cells between sequential frames from new adhesion events and quantifies the total time spent by a cell on the surface before detachment.

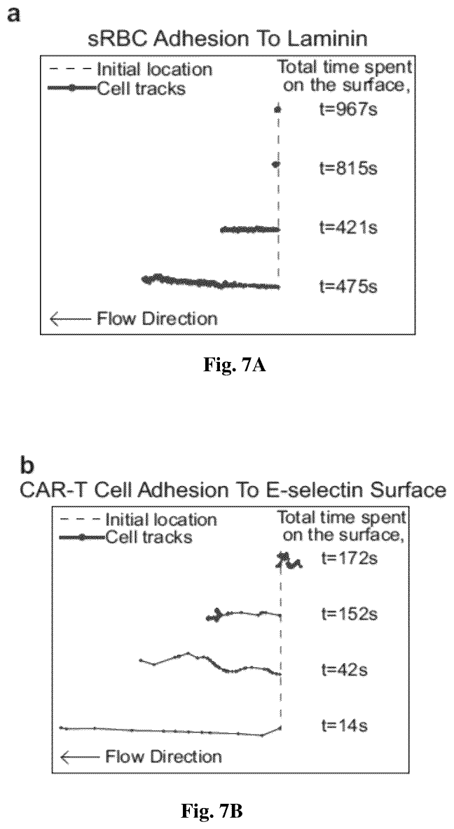

We show eight representative cell trajectories in FIGS. 7(A,B) four for sRBC adhesion to laminin, and four for CAR-T cell adhesion to E-selectin. We highlight three distinct types of adhesion events: adhesion events with large displacements, adhesion events with small displacements in the same direction as the flow, and adhesion events with small displacements in the opposite direction of the flow. These trajectories are generated for each adhered cell in an MBM video, giving us a high-throughput approach to collect dynamical information about large numbers of cells.

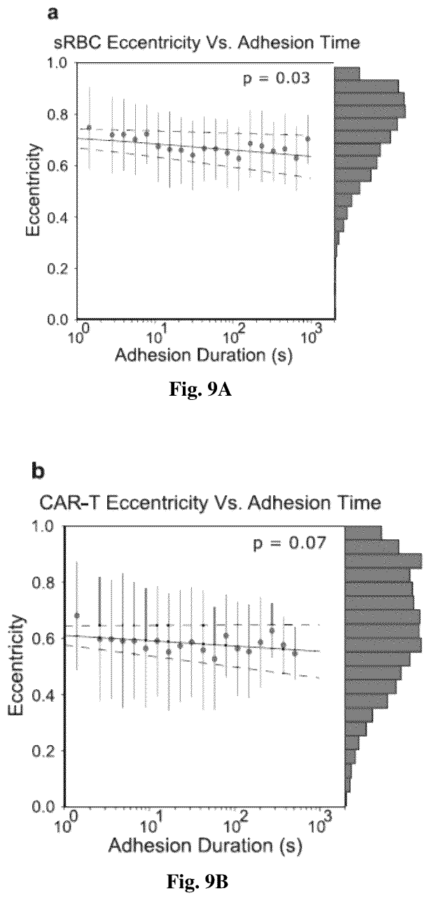

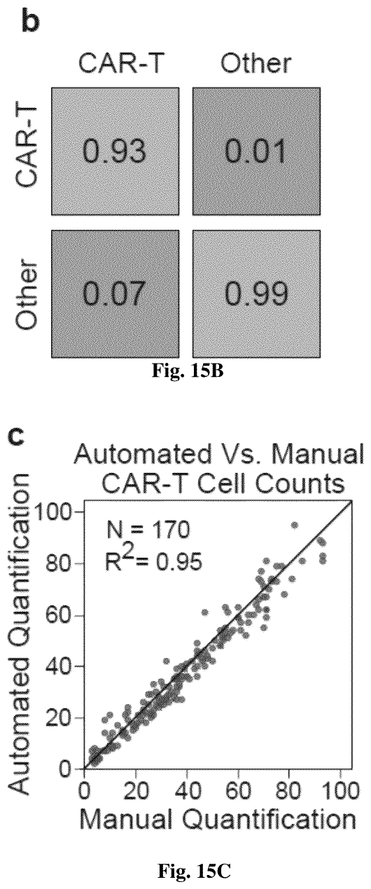

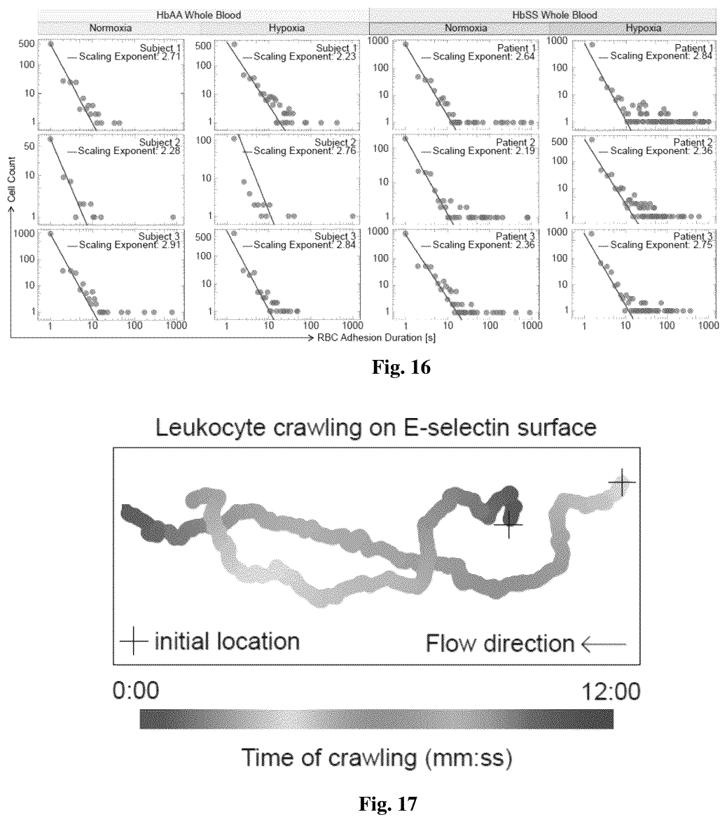

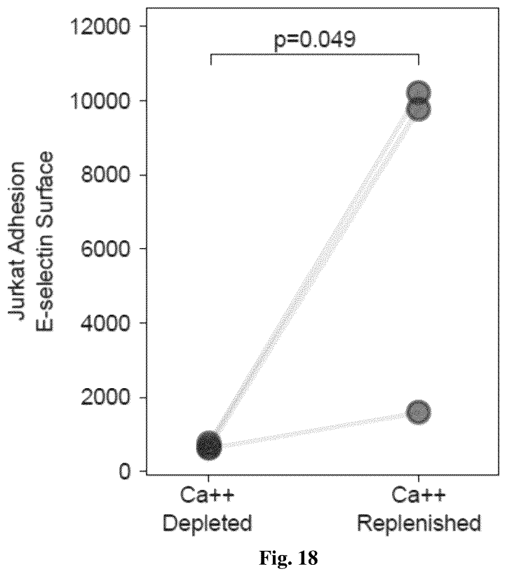

The adhesion duration distributions in FIG. 8 are one example of the dynamical data that can be compiled through our method, corresponding to thousands of individual trajectories. FIG. 8A shows sRBC adhesion to laminin, and FIG. 8B shows CAR-T cell adhesion to E-selectin. In both cases there is a peak in the distribution at low adhesion durations, corresponding to a large number of rapid attachment/detachment events, but also a non-trivial proportion of long-lived trajectories. At intermediate times, both plots are approximately power-law. At long times, the sRBC case continues the power-law trend, while CAR-T cells experience more rapid decay. The power law trend also applied to HbAA RBCs from healthy donors without known hemoglobinopathies for short-lived RBC adhesion events and was independent of oxygenation conditions for both HbSS and HbAA RBCs (FIG. 16). The longest adhesion durations (hundreds of seconds) occur infrequently, but the number of events collected by MBM is sufficiently large to resolve these rare cases. Adhesion duration distributions are essential raw data for biophysical modeling of bond dynamics between cells and surface proteins under flow conditions, providing a valuable starting point for future studies. MBM also has the potential of revealing more subtle dynamical relationships that have not been systematically explored, for example how morphological features of cells are correlated with dynamical behaviors at the single-cell level. A longstanding question about sRBCs is whether elongated, irreversibly sickled cells are more adhesive to endothelium than sickle discocytes. In FIG. 9A, we show MBM data for the relationship between average sRBC eccentricity (calculated over the entire cell trajectory) and adhesion duration to laminin, revealing a significant negative trend (p<0.03, linear regression): cells with longer adhesion durations are slightly less elongated on average. In FIG. 9B, we show that such a trend is not statistically significant between average eccentricity and adhesion duration to E-selectin for CAR-T cells (p>0.07, linear regression).

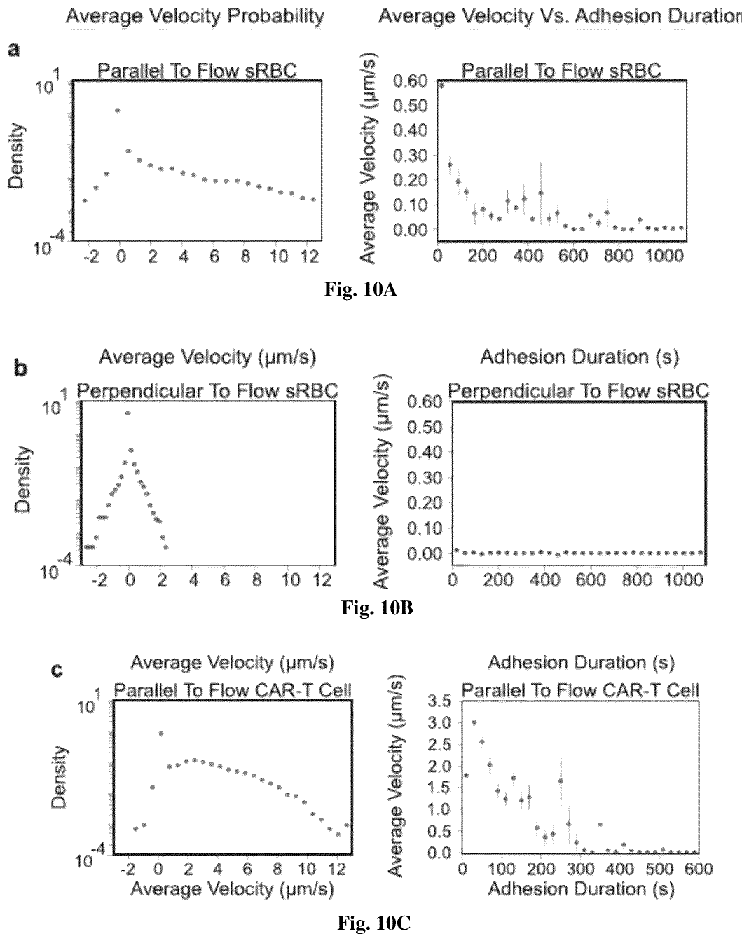

We can also investigate how the orientation of the cell motion relative to the flow direction influences the dynamics. For each cell trajectory, the initial and final position (before detachment) defines a net displacement, which divided by adhesion duration gives us an average velocity vector. The left column of FIG. 10 shows distributions of the components of this vector parallel to and perpendicular to the flow direction. Moreover, we can correlate the average velocity components with adhesion duration (right column of FIG. 10).

This analysis reveals a variety of interesting features. The left panel of FIG. 10A shows sRBC velocities on laminin, projected parallel to the flow direction. The distribution is peaked at zero velocity but has contributions from both positive (with flow) and negative (against flow) velocities. As expected, the distribution is distinctly asymmetric: cells are less likely to move against the flow. The right panel of FIG. 10A bins the sRBCs by adhesion duration and depicts the average parallel velocity for each bin. The shortest adhesion durations have the largest velocities, in agreement with the sample trajectories of FIG. 7A. For velocities perpendicular to the flow direction (FIG. 10B, left) the asymmetry in the distribution vanishes: average velocities in either the up or down perpendicular direction are equally likely. The large tail at positive velocities that was visible in the parallel distribution also disappears. In order to achieve velocities with magnitudes much greater than 2 μm/s one clearly requires the assistance of flow in the same direction as the cell motion.

FIG. 10C analyzes the motility of CAR-T cells adhered to E-selectin, parallel to the flow direction. We noted in FIG. 7 that CAR-T cells appeared to be more mobile than sRBCs, and this characteristic is validated in the distribution of average velocities (FIG. 9c, left). Relative to the velocity distribution of sRBCs (FIG. 10A, left), we see about an order of magnitude higher probabilities at short-intermediate positive velocities. In fact, there is a subsidiary peak in the CAR-T distribution around 2 μm/s, in addition to the zero-velocity peak. This trend is also evident when looking at the correlation of velocity with adhesion duration (FIG. 10C, right). On top of the overall trend of shorter adhesion durations associated with higher velocities, we see that CAR-T cells experience an order of magnitude higher velocity at short-intermediate adhesion durations relative to sRBCs.

FIG. 10 is just one illustration of the versatility of MBM as an in vitro platform for blood cell motility analysis, which to date has been exclusively performed with intravital microscopy. Another promising area for exploration is the impact of environmental signals on motility, for example, CD19 activation of CAR-T cells. The majority of CAR-T cells evaluated for B-cell malignancies target CD19. T cells express programmed cell death protein 1 (PD-1) during activation and intravital microscopy can show that T cell motility is proportional to PD-1 expression. As an in vitro alternative, we compare CAR-T velocities on E-selectin (parallel to the flow) with and without CD19 activation by using MBM (FIG. 11). FIG. 11a shows the velocity distribution for unactivated cells as a control reference, the same distribution as in FIG. 10C but plotted on a linear scale. The velocity bins are color-coded by different velocity regimes. In the absence of CD19 activation, adhered cells fall mainly into relatively immobile populations (the peak around zero velocity, highlighted in purple) and those moving at below 2 m/s (light blue). As expected, the small velocity population consists mainly of cells that adhere at one location, while the mobile cells show a range of trajectory lengths. CD19 activation significantly enhances the mean velocity of CAR-T cells rolling on E-selectin (FIG. 10B, C, p<0.001, t-test). We also use the same color scale to label and show the trajectories in FIG. 11D, E by their respective velocity regimes. Notably, more complicated motile leukocyte behavior such as crawling can also be captured with MBM (FIG. 17).

MBM enables researchers to investigate the dynamics of cellular interactions in the presence of whole blood flow. This means that many of the relevant physical forces and biochemical signals which modulate cellular interactions will be present during an experiment. Environmental completeness would be especially crucial when the functional pathways of cells are actively regulated by their environment. One example of such regulation is the influence of the complement and renin-angiotensin systems on leukocyte function. Therefore, we anticipate that MBM will have a formative effect on microfluidic studies. Even so, a new method is only as good as the available tools for analysis. Our machine learning pipeline for MBM can automatically analyze hundreds of thousands of observed adhesion events. Typically, the task of completing this analysis manually would be impossible, as the amount of time required, or the number of people required, would be far too large. Our automated analysis approach is flexible and mitigates errors due to individual human biases in cell counting and classification.

The applications of MBM are wide-ranging for both fundamental investigations into biophysical mechanisms and clinical studies. For example, adhesion duration distributions are crucial for studies of force-dependent binding/unbinding of protein complexes and cell-cell interactions. MBM can complement existing approaches in this area like numerical simulations or force spectroscopy experiments (i.e., atomic force microscopy). MBM could provide a valuable comparison point for bond lifetime simulations, adding extra physiological realism due to the blood flow. Because of its ease of implementation, it is also accessible to a broader research community than atomic force microscopy techniques. On the clinical front, there are a variety of promising applications for specific diseases. In the case of cancer, MBM could help visualize crucial interactions of circulating tumor and endothelial cells with improved physiological accuracy. It can also be utilized to distinguish and isolate rare circulating tumor cells from billions of other cells flowing through a microfluidic channel. For sickle cell disease, MBM could serve as a means to understand how sRBCs behave under changes in blood pressure, flow rates, and viscosity, as well as concentrations of relevant constituents in whole blood. Furthermore, the concentration of adhered sRBCs, as well as the morphological characteristics of the cells may be used to monitor if an individual with sickle cell disease is undergoing a flare-up, or if they are in an asymptomatic diseased state. Other possible disease contexts where MBM may be useful are solid tumor malignancies. MBM may help us characterize the motility of immune cells including T-cells and macrophages with great physiological relevance. As a final example, MBM could be used to further our understanding of autoimmune diseases, such as rheumatoid arthritis, where leukocyte recruitment is an important part of the pathology. For example, MBM can be used in tandem with joint on a chip systems, where complex interactions among blood cells, endothelium, and skeletal components are studied with in vitro experiments.

Aside from clinical studies, MBM with automated analysis has the ability to efficiently gather large quantities of data that may provide an ideal basis for theoretical modeling of cellular adhesion mechanics. For example, the velocity distributions parallel to the flow direction from FIG. 10 qualitatively resemble those of molecular motors that are capable of forward and backward stepping along a cytoskeletal track, which can be described via coarse-grained kinetic models of the underlying biochemical cycle. Similar mathematical approaches could be brought to bear on the cell velocity data, giving us a more complete picture of the cell interactions with the endothelium that give rise to these kinds of dynamics. The excellent statistics of the data set is crucial in this regard since in principle it enables us to distinguish between competing models.

As with any experimental approach, there are also limitations. In sufficiently complex scenarios where large densities and/or multiple cell types adhere to the surface, extracting single-cell dynamical information may become more difficult. Large densities, with many overlapping adhered cells, could impede the video analysis in determining if two cells in consecutive frames are the same or not. Overlaps were rare in the examples we investigated, allowing for straightforward extraction of cell trajectories. In physical systems where this would be an issue, however, more sophisticated methods could be incorporated into MBM analysis workflow for tracking cells. Similarly, both the sRBC and CAR-T datasets we focused on in the main text involve a single adhered cell type, which could be identified reliably using a size threshold. In cases with multiple cell types of similar sizes, we would have to employ a more involved classification procedure, using other morphological characteristics of the cells. For example, a convolutional neural network was trained to reliably distinguish CAR-T cell adhesion to P-selectin from red blood cells, which also adhere to the surface. Thus we believe all these limitations may be substantially addressed with additions to the data analysis approach. Extended exposure time inherently offsets the eccentricity estimations of the interacting cells. The magnitude of the offset would depend on the velocity of the cells. Eccentricity estimation of high motility cells including T-cells may be significantly affected by this issue. In a typical scenario, a perfectly round CAR-T cell with a size of 8 am, moving with a mean velocity of 2 m/s would travel 2.4 am during the 1200 ms exposure time of the camera. In this case, the resulting eccentricity estimate would be offset by 0.32. This effect can be accounted for using a velocity-based calibration curve for the major axis a, during the eccentricity e calculation:

$$e = \sqrt{1 - {b}^{2} / {a}_{corr}^{2}} \qquad ( 1 )$$

where b is the minor axis length and the corrected major axis length a_{corr} would be given by:

$${a}_{corr} = {a}_{meas} - {V}_{cell} {t}_{exp′} \qquad ( 2 )$$

where V_{cell} is the velocity parallel to flow, texp is the exposure time of the camera, and aₘₑₐₛ is the measured major axis length. Strictly speaking, the major axis might not align entirely with the velocity vector, in which case, the calculation would become more complicated. Even so, we believe this simple adjustment should mostly account for changes in eccentricity due to flow velocity. We performed eccentricity offset calibration based on velocity for CAR-T cell adhesion to E-selectin in FIGS. 4 and 8.

In summary, MBM is a robust, easy-to-implement, high-throughput method to study cell adhesion dynamics in the presence of blood flow. Its flexibility allows for broad deployment in both clinical contexts and studies of fundamental biophysical mechanisms underlying cell adhesion. Combined with automated analysis for cell classification and tracking, it promises to be a general platform for elucidating how interactions with the complex whole blood environment influence and regulate cellular adhesion and interactions.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

Having described the invention, we claim:

1. A system comprising:
a microfluidic device having at least one microchannel through which a fluid sample including cells flows;
an imaging system configured for generating one or more sets of motion blur microscopy (MBM) images of cells of interest in the microchannel when the fluid sample containing cells is passed therethrough;
one or more non-transitory computer-readable storage media including instructions; and
one or more processors coupled to the one or more non-transitory computer-readable storage media, the one or more processors configured to execute the instructions to:
access the set of one or more MBM images generated by the imaging system;
input the set of the one or more MBM images into one or more machine learning models trained to:
generate a segmentation mask based on based on the set of one or more MBM images, the segmentation mask including a adhered pixels or background pixels, the adhered pixels corresponding to a pixel of the MBM images belonging to an adhered object in the microchannel and with remaining pixels being background pixels; and
generate a cell classification of the adhered pixels based on a physical property of the cells; and
detect type or hemodynamic properties of the cells based on the cell classification.

2. The system of claim 1, wherein the microfluidic device includes a housing with at least one microchannel defining at least one cell adhesion region, the at least one cell adhesion region being provided with at least one capturing agent that adheres a cell of interest to a surface of the at least one microchannel when a fluid sample containing cells is passed through the at least one microchannel.

3. The system of claim 2, wherein the imaging system includes a camera configured to obtain a plurality of MBM images of the fluid sample flowing through the cell adhesion region.

4. The system of claim 3, wherein processor is configured to identify groups of pixels in the MBM image corresponding to adhered cells and generate of the cell classification based cell size.

5. The system of claim 4, wherein the cell classification determines the type of cell.

6. The system of claim 5, wherein the one or more machine-learning models include a segmentation model and a classification model.

7. The system of claim 6, wherein the segmentation model is trained by selecting one or more MBM images as training/validation images, creating a labeled training/validation mask of the training/validation images by labeling each pixel as adhered or background, splitting each training/validation mask and training/validation images into tiles of pixels, and applying data augmentation to produce unique orientations of the tiles.

8. The system of claim 7, wherein the segmentation model labels pixels from the MBM images corresponding to adhered objects and groups together neighboring labeled pixels.

9. The system of claim 7, wherein the processor uses groups of labeled pixels to classify the cell type using a size threshold or a (specifically trained classification neural network.

10. The system of claim 7, wherein the processor further classifies cell type by using cell morphological properties as well as cell dynamic properties.

11. The system of claim 10, wherein morphological properties cells size and eccentricity and the cell dynamic properties include cell adhesion duration or mean velocity.

12. A method of determining one or more hemodynamic properties or cell type of a blood sample, the method comprising:
obtaining a set of one or more motion blur microscopy (MBM) images of the blood sample flowing through a microchannel of a microfluidic device; and
inputting the set of one or more MBM images into one or more machine learning models trained to:
generate a segmentation map based on based on the set of one or more MBM images, the segmentation map including a adhered pixels or background pixels, the adhered pixels corresponding to a pixel of the MBM images belonging to an adhered object in the microchannel and with remaining pixels being background pixels; and
generate a cell classification of the adhered pixels based on a physical property of the cells; and
detect type or hemodynamic properties of the cells based on the cell classification.

13. The method of claim 12, wherein the microfluidic device includes a housing with at least one microchannel defining at least one cell adhesion region, the at least one cell adhesion region being provided with at least one capturing agent that adheres a cell of interest to a surface of the at least one microchannel when a fluid sample containing cells is passed through the at least one microchannel.

14. The method of claim 13, wherein machine learning model is configured to identify groups of pixels in the MBM image corresponding to adhered cells and generate a cell classification based cell size.

15. The method of claim 14, wherein the cell classification determines the type of cell.

16. The method of claim 15, wherein the one or more machine-learning models include a segmentation model and a classification model.

17. The method of claim 16, wherein the segmentation model is trained by selecting one or more MBM images as training/validation images, creating a labeled training/validation mask of the training/validation images by labeling each pixel as adhered or background, splitting each training/validation mask and training/validation images into tiles of pixels, and applying data augmentation to produce unique orientations of the tiles.

18. The method of claim 17, wherein the segmentation model labels pixels from the MBM images corresponding to adhered objects and groups together neighboring labeled pixels.

19. The method of claim 17, wherein the machine learning model uses groups of labeled pixels to classify the cell type using a size threshold or a specifically trained classification neural network.

20. The system of claim 17, wherein the classification model further classifies cell type by using cell morphological properties as well as cell dynamic properties.
